(19) United States
(12) Patent Application Publication
(10) Pub. No.: US 2026/0252962 A1
(43) Pub. Date: Aug. 27, 2026

(54) SYSTEM

(71) Applicant: SoftBank Group Corp., Tokyo (JP)

(72) Inventor: Yuki ICHIOKA, Tokyo (JP)

(73) Assignee: SoftBank Group Corp., Tokyo (JP)

(21) Appl. No.: 19/539,294

(22) Filed: Feb. 13, 2026

(30) Foreign Application Priority Data
Feb. 21, 2025 (JP) 2025-027074

(51) Int. Cl. G06N 20/00

(52) U.S. Cl. CPC G06N 20/00

(57) ABSTRACT

The system according to the embodiment comprises a reception unit, a generation unit, an analysis unit, a proposal unit, a management unit, and an image generation unit. The reception unit receives a training purpose. The generation unit generates a personal training menu based on the purpose received by the reception unit. The analysis unit analyzes an exercise form based on the menu generated by the generation unit. The proposal unit proposes a meal plan based on the menu generated by the generation unit. The management unit performs progress management based on the menu generated by the generation unit. The image generation unit presents an appearance of a user before and after achieving the purpose using an image generation function.

### CROSS-REFERENCE TO RELATED APPLICATION

The present application claims priority to and incorporates by reference the entire contents of Japanese Patent Application No. 2025-027074 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, provision of a personal training menu according to a training purpose and progress management have not been sufficiently performed, and there is room for improvement.

### SUMMARY OF THE INVENTION

The system according to the embodiment comprises a reception unit, a generation unit, an analysis unit, a proposal unit, a management unit, and an image generation unit. The reception unit receives a training purpose. The generation unit generates a personal training menu based on the purpose received by the reception unit. The analysis unit analyzes an exercise form based on the menu generated by the generation unit. The proposal unit proposes a meal plan based on the menu generated by the generation unit. The management unit performs progress management based on the menu generated by the generation unit. The image generation unit presents an appearance of a user before and after achieving the purpose using an image generation function.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

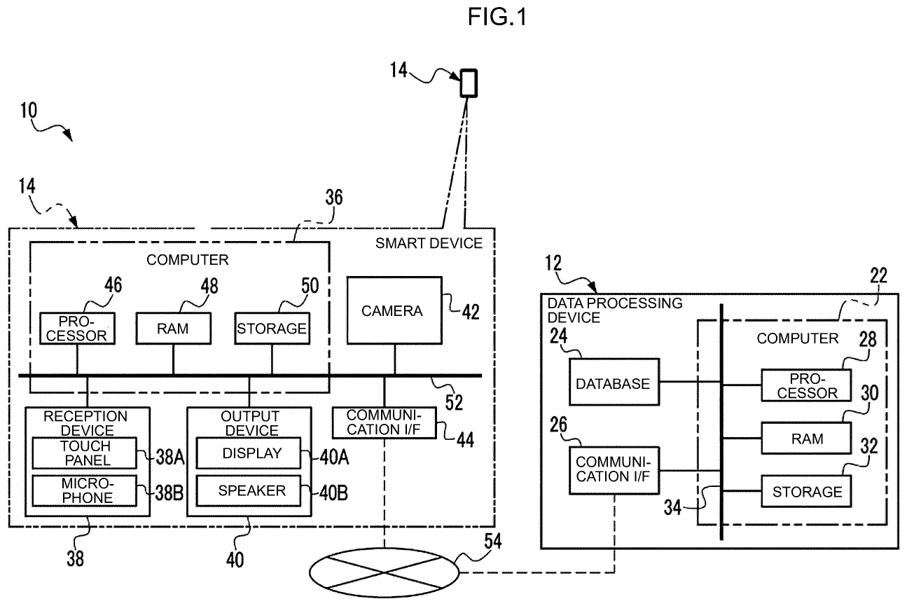

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

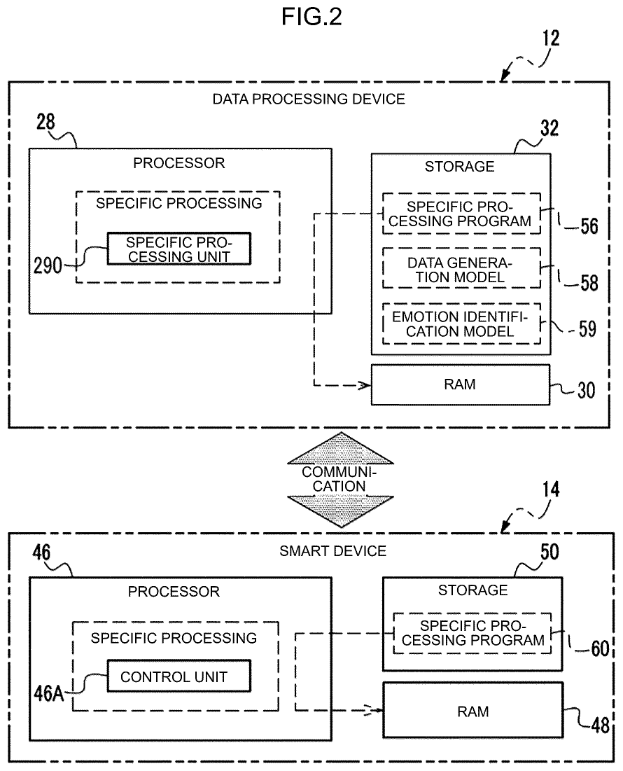

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The personal training system according to the embodiment of the present invention is a system that provides personal training tailored to purposes such as health, diet, and muscle strength improvement. In this personal training system, a user inputs a training purpose, and the system generates a personal training menu based on the user's purpose. This menu includes analysis of an exercise form, proposal of a meal plan optimal for achieving a goal, progress management, and the like. Also, an “appearance of oneself” before and after achieving the purpose can be presented using an image generation function. For example, a user inputs a training purpose. For example, the user inputs a purpose such as diet or muscle strength improvement. This information is input to the system. Next, the system generates a personal training menu based on the input purpose. The system proposes an exercise menu according to the user's purpose. For example, for a user whose purpose is muscle strength improvement, a menu for muscle training is proposed. Furthermore, the system performs analysis of an exercise form. When the user performs an exercise, the system analyzes the form and checks whether the exercise is being performed with an appropriate form (correct form). Thereby, the user can perform the exercise with the correct form, enabling effective training. Also, the system proposes a meal plan optimal for achieving a goal. The system generates a meal plan according to the user's purpose and provides it to the user. For example, for a user whose purpose is diet, a meal plan including calorie restriction is proposed. Furthermore, the system performs progress management. The system manages progress of the user's training and provides support for achieving a goal. For example, the system records training results and provides feedback to the user. Finally, the system presents the “appearance of oneself” before and after achieving the purpose using the image generation function. The system generates images of the appearance before and after the user starts the training, and presents them to the user. Thereby, the user can increase motivation for achieving the goal. Thereby, the personal training system enables generation of a personal training menu according to the user's training purpose, analysis of an exercise form, proposal of a meal plan, progress management, and image generation. Specifically, the present system is implemented as an advanced information processing platform integrating a plurality of deep learning models operating on a distributed cloud computing environment and edge devices. The present system comprises a natural language processing module for analyzing input in natural language by the user, a computer vision module for analyzing body motion in real time, and an image generation module including a Generative Adversarial Network (GAN) or a Diffusion Model for predicting and visualizing future physical changes. For example, text data such as “I want to get six-pack abs by summer” input by the user is converted into a high-dimensional vector representation (embedding vector) by a Transformer-based language model such as BERT or GPT, and the user's intention (intent) and extracted parameters (period, body part, target intensity) are stored in a database as structured data. Based on this structured data, a recommendation engine using a reinforcement learning algorithm (e.g., Deep Q-Network or PPO) dynamically generates a training menu optimal for the user's current physical condition and goal from among millions of exercise combinations. In the analysis of the exercise form, RGB image frames (e.g., a video stream with a resolution of 1920×1080, 30 fps) acquired from the user's smartphone or Web camera are input to a Convolutional Neural Network (CNN), and two-dimensional or three-dimensional coordinates of major joint points (key points) of the human body are estimated in real time. Time series data of the estimated joint coordinates is compared with coordinate data of an ideal form using Dynamic Time Warping (DTW) or the like, and a score based on cosine similarity or Euclidean distance is calculated, whereby the quality of the form is quantitatively determined. Furthermore, in the image generation function, a current whole-body image of the user is used as input, and by performing an operation in a latent space using body shape parameters after achieving the goal (e.g., 10% decrease in body fat percentage, 5 kg increase in muscle mass) as a conditioning vector, a photorealistic and high-definition image of “future self” is generated. In this way, the present system provides a technical foundation that comprehensively supports the user's physical and psychological transformation by making full use of multimodal AI technology, not merely presenting information.

The personal training system according to the embodiment comprises a reception unit, a generation unit, an analysis unit, a proposal unit, a management unit, and an image generation unit. The reception unit allows a user to input a training purpose. For example, the user inputs a purpose such as diet or muscle strength improvement. The generation unit generates a personal training menu based on the purpose received by the reception unit. For example, the generation unit generates an exercise menu according to the user's purpose. For example, the generation unit generates a menu for muscle training for a user whose purpose is muscle strength improvement. The analysis unit analyzes an exercise form based on the menu generated by the generation unit. For example, the analysis unit analyzes the form when the user performs an exercise, and checks whether the exercise is being performed with an appropriate form (correct form). The proposal unit proposes a meal plan based on the menu generated by the generation unit. For example, the proposal unit generates a meal plan according to the user's purpose and provides it to the user. The management unit performs progress management based on the menu generated by the generation unit. For example, the management unit manages progress of the user's training and provides support for achieving a goal. The image generation unit presents an “appearance of oneself” before and after achieving the purpose using an image generation function. For example, the image generation unit generates images of the appearance before and after the user starts the training, and presents them to the user. Thereby, the user can increase motivation for achieving the goal. Thereby, the personal training system according to the embodiment enables generation of a personal training menu according to the user's training purpose, analysis of an exercise form, proposal of a meal plan, progress management, and image generation. Specifically, each unit of the present system is deployed as an independent container based on a microservice architecture, and is configured as a distributed processing system that cooperates with each other through an API gateway. The reception unit receives input from a user interface (UI), performs text normalization and speech-to-text (STT) conversion as preprocessing, and then transmits a request to a subsequent processing unit together with a user ID and session information. The generation unit combines a user profile (age, sex, exercise history, BMI, etc.) and the input purpose as a feature vector, inputs it to a prediction model using a Multilayer Perceptron (MLP) or a Recurrent Neural Network (RNN), thereby outputting a recommendation probability (score from 0 to 1) of each exercise item, and constructs a menu by combining items with high scores. The analysis unit may adopt a hybrid configuration in which edge computing technology is utilized to execute a lightweight skeleton estimation model (e.g., a model having a MobileNet backbone) on the user terminal side to protect privacy, and only extracted feature point data is transmitted to a server for detailed analysis. The proposal unit comprises an optimization solver that cooperates with a nutrient database and uses linear programming or a genetic algorithm to search for a meal plan closest to the user's preference while satisfying constraints of calorie restriction and PFC balance (protein, fat, carbohydrate). The management unit accumulates daily training logs and vital data using a Time Series Database, and performs future result prediction or detection of a plateau period using a time series prediction model such as LSTM (Long Short-Term Memory). The image generation unit uses a large-scale image generation model operating on a GPU cluster, performs an operation of adding or subtracting an attribute vector corresponding to a target body shape to or from a latent feature extracted from the user's current image, and reconstructs an image through a decoder, thereby generating a physically consistent and high-precision simulation image. With these configurations, the present system produces a technical effect of providing a highly personalized training experience to each user with low latency and high reliability.

The generation unit can generate an exercise menu according to the user's purpose. The generation unit generates, for example, an exercise menu according to the user's purpose. For example, the generation unit generates a menu for muscle training for a user whose purpose is muscle strength improvement. Also, the generation unit can generate an exercise menu emphasizing calorie consumption for a user whose purpose is diet. Furthermore, the generation unit can generate a balanced exercise menu for a user whose purpose is health maintenance. Thereby, the generation unit generates an exercise menu according to the user's purpose, enabling effective training. Specifically, the present generation unit comprises an inference engine that executes a hybrid recommendation algorithm combining collaborative filtering and content-based filtering. Input to the generation unit is a multidimensional tensor including a user attribute vector (age, sex, weight, body fat percentage, etc.), a purpose vector (one-hot encoding or embedding representation of muscle hypertrophy, weight loss, endurance improvement, etc.), and past training history data (item, number of times, number of sets, subjective exercise intensity RPE). The generation unit inputs these input data to a neural network, and outputs a “fitness score” and a “recommended number of sets/times” for each of thousands of exercise items. For example, when the purpose is muscle strength improvement, the generation unit outputs a high fitness score (e.g., 0.95) for high-load, low-repetition items (bench press, squat, etc.) effective for muscle hypertrophy, and outputs a low score (e.g., 0.20) for aerobic exercise. Furthermore, in order to consider the user's fatigue level and recovery status, the generation unit learns a time-series context using a Recurrent Neural Network (RNN) or Transformer, and dynamically adjusts an optimal exercise intensity on a specific day. For example, if intense leg training was performed on the previous day, the generation unit performs suppression control such as lowering the score of leg items and raising the score of upper body items. The output menu data is transmitted to the client terminal as structured data such as JSON format, and is visualized on the UI. In this way, the present generation unit has a technical effect of generating an optimal and safe training menu that immediately responds to a change in the user's state by performing probabilistic inference based on multidimensional data, rather than a static rule base.

The analysis unit can analyze the form when the user performs an exercise, and confirm whether the exercise is being performed with an appropriate form. The analysis unit analyzes, for example, the form when the user performs an exercise, and confirms whether the exercise is being performed with an appropriate form. For example, the analysis unit analyzes the user's posture and smoothness of motion, and checks whether the exercise is being performed with a correct form. Also, the analysis unit can analyze the user's exercise form in real time and point out corrections for the form. Furthermore, the analysis unit can record the user's exercise form and analyze it in detail later. Thereby, the analysis unit checks whether the exercise is being performed with a correct form, enabling effective training. Specifically, the present analysis unit is configured by an image processing processor that executes a pose estimation algorithm based on deep learning. Input to the analysis unit is continuous image frames (e.g., RGB image tensor: height H×width W×3 channels) acquired from a camera. The analysis unit applies a CNN (e.g., a model having ResNet or EfficientNet as a backbone) to each frame, and outputs two-dimensional coordinates (x, y) of major joints of the human body (total 17 to 33 points such as shoulder, elbow, wrist, waist, knee, ankle) and confidence scores thereof. Furthermore, in order to perform time-series motion analysis, the analysis unit inputs a sequence of the extracted joint coordinates to an LSTM or a Temporal Convolutional Network (TCN), and calculates a cycle, velocity, acceleration, and a change pattern of joint angles of the motion. Determination of whether the form is correct is performed using distance calculation by Dynamic Time Warping (DTW) between an “ideal trajectory model” learned from motion data of experts and the user's “actual trajectory”, or an anomaly detection algorithm (e.g., One-Class SVM or reconstruction error of an autoencoder). For example, if the knee protrudes too far forward than the toe in a squat motion, the analysis unit detects a deviation from a relative positional relationship between the knee joint and the ankle joint, and outputs a specific feedback signal (text, voice, or visual indicator on a screen) such as “Knees are too far forward”. This processing is executed with low latency (e.g., within 30 ms) per frame, realizing real-time feedback to the user. Thereby, technical improvement is made to maximize the training effect while reducing the risk of injury.

The proposal unit can generate a meal plan according to the user's purpose and provide it to the user. The proposal unit generates, for example, a meal plan according to the user's purpose and provides it to the user. For example, the proposal unit generates a meal plan including calorie restriction for a user whose purpose is diet. Also, the proposal unit can generate a meal plan containing a lot of protein for a user whose purpose is muscle strength improvement. Furthermore, the proposal unit can generate a balanced meal plan for a user whose purpose is health maintenance. Thereby, the proposal unit generates and provides a meal plan according to the user's purpose, enabling support for achieving a goal. Specifically, the present proposal unit is configured by a mathematical optimization engine and a recommendation algorithm that search for an optimal solution satisfying nutritional constraints. Input to the proposal unit is user profile data including the user's Basal Metabolic Rate (BMR), activity level, target weight, allergy information, and preference data (liked ingredients, disliked ingredients). The proposal unit first calculates a target calorie intake per day and a target ratio of macronutrients (protein, fat, carbohydrate) using a calculation formula such as the Harris-Benedict equation. Next, the proposal unit executes optimization processing formulated as a knapsack problem or a Constraint Satisfaction Problem (CSP) on a database containing tens of thousands of recipe data. At this time, an objective function is defined as “maximization of user preference score” and “minimization of error from target nutrients”. For example, when the purpose is muscle strength improvement, high weighting is given to recipes having a protein content equal to or higher than a threshold (e.g., 30 g/meal), and an optimal menu set (breakfast, lunch, dinner, snack) whose total calories fall within a target range is selected from among combinations thereof. Output data is structured data (JSON, etc.) including a menu name, an ingredient list, a nutrient breakdown, and a recipe procedure for each meal. Furthermore, the proposal unit receives a record of meals actually ingested by the user (by image recognition or text input) as feedback, and performs dynamic recalculation to compensate for missing nutrients within the remaining allowable calories in the next proposal. Thereby, adaptive meal management that responds immediately to the user's real life is realized, rather than a static menu table.

The management unit can manage progress of the user's training and provide support for achieving a goal. The management unit manages, for example, progress of the user's training and provides support for achieving a goal. For example, the management unit records training results of the user and grasps the progress status. Also, the management unit can adjust the training menu according to the progress of the user's training. Furthermore, the management unit can provide feedback to the user and provide support for increasing motivation for achieving the goal. Thereby, the management unit manages progress of the user's training, enabling support for achieving a goal. Specifically, the present management unit is a data processing module comprising a time series data analysis engine and adaptive control logic. Input to the management unit is multivariate time series data such as daily training results (lifted weight, number of times, running distance, etc.), physical measurements (weight, body fat percentage, muscle mass), and subjective fatigue level. The management unit accumulates these data in a database, and performs trend analysis using a moving average or exponential smoothing, or future prediction using an ARIMA model or an LSTM network. For example, the management unit analyzes a weight loss trend for the past month, and predicts and outputs a scheduled goal achievement date. If the predicted progress deviates from the goal (e.g., weight loss is stagnant), the management unit detects a “plateau”, and transmits a command signal (feedback control signal) to the generation unit to change the training load (intensity coefficient). Also, the management unit performs scoring processing incorporating gamification elements in order to encourage user behavior modification. For example, “experience points” or “badges” are granted based on the number of continuous training days or goal achievement level, and these are visualized and displayed on the user terminal. Furthermore, the management unit also has a function of detecting a pattern suggesting a health risk such as rapid weight loss or excessive training frequency using an anomaly detection algorithm, and outputting a warning alert. In this way, the management unit plays a technical role of guiding the user to goal achievement via the shortest route by performing prediction and control based on data, not merely as a recorder.

The image generation unit can generate images of an appearance before and after the user starts the training, and present them to the user. The image generation unit generates, for example, images of an appearance before and after the user starts the training, and presents them to the user. For example, the image generation unit photographs an appearance before the user starts the training, and predicts and generates an appearance after the training based on the image. Also, the image generation unit can display a change in appearance in real time according to the progress of the user's training. Furthermore, the image generation unit can generate before-and-after images for comparing changes in the user's appearance. Thereby, the image generation unit generates and presents images of the appearance before and after the user starts the training, enabling increasing motivation for achieving the goal. Specifically, the present image generation unit is a GPU accelerated computing module implementing a deep generative model such as a Generative Adversarial Network (GAN), a Variational Autoencoder (VAE), or a Diffusion Model. Input to the image generation unit is a current whole-body image (source image) of the user and parameters representing target physical characteristics (target attributes: e.g., “body fat percentage 15%”, “muscle mass +5 kg”, “waist −3 cm”). The image generation unit first passes the source image through an encoder to convert it into a latent vector in a latent space. Next, based on the target attributes, a semantic operation (vector operation) is performed on this latent vector. For example, a direction vector corresponding to “muscle mass” is identified in the latent space, and the user's latent vector is moved in that direction. Thereafter, by passing the manipulated latent vector through a decoder (generator), a synthetic image in which only the body shape has changed to the target state while maintaining the identity (face, hairstyle, background, etc.) of the original person is generated. Output data is high-resolution RGB image data. Furthermore, the image generation unit can generate a plurality of images of intermediate processes from the current state to the target state, and generate a morphing video showing how the body gradually changes by continuously playing them. With this technology, the user can confirm not only numerical data but also a visual and intuitive future prediction map, and obtain strong motivation for continuing long-term training.

The reception unit can estimate an emotion of the user, and adjust an input method of the training purpose based on the estimated emotion of the user. The reception unit estimates, for example, an emotion of the user, and adjusts an input method of the training purpose based on the estimated emotion of the user. For example, when the user feels stress, the reception unit provides a simple interface to minimize input procedures. Also, when the user is relaxed, the reception unit can provide detailed input options and propose a customizable input method. Furthermore, when the user is in a hurry, the reception unit can prioritize voice input to enable quick input of the training purpose. Thereby, the reception unit adjusts the input method of the training purpose based on the user's emotion, enabling provision of an input method optimal for the user. Estimation of the emotion is realized using an emotion estimation function using, for example, an emotion engine or generative AI. The generative AI is text generation AI (e.g., LLM) or multimodal generation AI, but is not limited to such examples. Specifically, the present reception unit functions as an intelligent interface control unit equipped with a multimodal emotion recognition model. Input to the reception unit is multimodal data including user's speech voice data (waveform, spectrogram), camera images (facial expression), and input text or keystroke patterns (typing speed, number of corrections). The reception unit extracts prosodic features (pitch, intensity, speech rate) from the voice data, detects movements of facial muscles (action units) from the image data, and analyzes emotion polarity (positive/negative) from the text data. These feature quantities are input to an integrated model such as a multimodal Transformer, and a probability distribution (emotion vector) representing the user's current emotional state (e.g., joy, anger, sadness, frustration, fatigue, relaxation) is output. For example, when it is determined that the score of “frustration” is high (e.g., 0.8 or more), the reception unit sends a control signal to a UI rendering engine to switch to a “simple mode” in which the number of input items on the screen is reduced, button sizes are enlarged, and a voice input mode is activated by default. On the other hand, when the scores of “relaxation” and “interest” are high, an “expert mode” allowing detailed parameter settings is displayed, and detailed hearing by an interactive agent is started. In this way, by dynamically reconfiguring the external interface according to the user's internal state (emotion), a technical effect of dramatically improving usability and input efficiency is produced.

The reception unit can analyze a past training history of the user, and propose a purpose input method. The reception unit analyzes, for example, a past training history of the user, and proposes an optimal purpose input method. For example, the reception unit automatically displays training purposes frequently input by the user in the past as candidates. Also, the reception unit can preferentially propose an input method (voice, text, etc.) used by the user in the past. Furthermore, the reception unit can predict and propose a training purpose to be used in a specific time zone from the user's past training history. Thereby, the reception unit analyzes the user's past training history, enabling proposal of an optimal purpose input method. Part or all of the above-described processing in the reception unit may be performed using, for example, AI, or may be performed without using AI. Specifically, the present reception unit comprises a sequence pattern mining or next action prediction model using the user's behavior log data as training data. Input to the reception unit is a group of records in a history database including past login dates and times, input purposes (labels), used input devices (keyboard, microphone, touch panel), and context information (day of week, time zone). The reception unit applies association analysis (Apriori algorithm, etc.), a Markov chain model, or a Recurrent Neural Network (RNN) to these data, and calculates a conditional probability of a “purpose” and an “input means” most likely to be selected by the user in the current context (e.g., Monday morning at 7:00). For example, if a pattern that “on weekday mornings, ‘light stretching’ is selected with a probability of 90% and voice input is used” is detected from past data, the reception unit displays a one-tap button “Do you want to do light stretching?” immediately after the app is launched, and simultaneously puts the microphone in a standby state. As output, a list of recommended purposes (list sorted in order of probability) and configuration parameters of a UI layout are generated. This significantly reduces the user's cognitive load and number of operation steps, and optimizes input efficiency to the system.

The reception unit can perform filtering based on a current health condition or lifestyle habits of the user when the training purpose is input. The reception unit performs, for example, filtering based on a current health condition or lifestyle habits of the user when the training purpose is input. For example, when the user's health condition is not good, the reception unit proposes a light training purpose. Also, the reception unit can propose an achievable training purpose based on the user's lifestyle habits. Furthermore, the reception unit can refer to the user's health data, and filter and display appropriate training purposes. Thereby, the reception unit filters training purposes based on the user's health condition or lifestyle habits, enabling proposal of achievable training purposes. Part or all of the above-described processing in the reception unit may be performed using, for example, AI, or may be performed without using AI. Specifically, the present reception unit comprises a filtering engine that cooperates with vital data (heart rate, blood pressure, sleep time, number of steps, SpO2, etc.) acquired from a wearable device or a healthcare app. Input to the reception unit is real-time vital values and interview data (pain, fatigue). The reception unit normalizes these values and performs comparison determination with thresholds set based on medical guidelines or individual baseline data. Alternatively, an anomaly detection model (Isolation Forest, etc.) is used to classify whether the current health condition belongs to “normal”, “caution”, or “danger”. For example, if the sleep time is less than 4 hours and the resting heart rate is higher than usual, the system executes filtering processing of excluding (graying out or hiding) high-load purposes such as “High Intensity Interval Training (HIIT)” from options, and activating only low-load purposes such as “yoga” and “stretching”. Output data is a “selectable purpose list” after applying filtering. With this function, a technical effect of preventing the user's reckless training selection in advance on the system side and automating health risk management is obtained.

The reception unit can estimate an emotion of the user, and determine a priority of the input training purpose based on the estimated emotion of the user. The reception unit estimates, for example, an emotion of the user, and determines a priority of the input training purpose based on the estimated emotion of the user. For example, when the user has high motivation, the reception unit preferentially displays a challenging training purpose. Also, when the user is tired, the reception unit can preferentially display a relaxing training purpose. Furthermore, when the user feels stress, the reception unit can preferentially display a training purpose useful for stress relief. Thereby, the reception unit determines the priority of the training purpose based on the user's emotion, enabling provision of a training purpose optimal for the user. Estimation of the emotion is realized using an emotion estimation function using, for example, an emotion engine or generative AI. The generative AI is text generation AI (e.g., LLM) or multimodal generation AI, but is not limited to such examples. Specifically, the present reception unit uses a Learning to Rank model incorporating an emotion score as a feature quantity. Input data is an emotion vector (e.g., [motivation: 0.9, fatigue: 0.1, stress: 0.2]) output from the aforementioned emotion estimation module, and an attribute vector of a candidate training purpose (e.g., [intensity: high, fun: medium, relaxation effect: low]). The ranking model (e.g., LambdaMART or neural ranking model) calculates an interaction between these vectors, and calculates a “fitness score” that each training purpose should be selected in the current user's emotional state. For example, when the emotion vector indicates “high stress”, scores of purposes having attributes with high stress relief effects such as “boxercise” and “meditation” are amplified and sorted to the top of the list. On the other hand, in the case of “high fatigue”, scores of high-intensity purposes are penalized and moved to the bottom. Output is a purpose list sorted in order of score, which is presented to the user on the UI. This proactively presents options matching the user's psychological state and supports decision making.

The reception unit can consider geographical location information of the user when the training purpose is input, and prioritize input of a highly relevant purpose. The reception unit considers, for example, geographical location information of the user when the training purpose is input, and prioritizes input of a highly relevant purpose. For example, when the user is near a gym, the reception unit preferentially displays training purposes that can be performed at the gym. Also, when the user is near a park, the reception unit can preferentially display training purposes that can be performed at the park. Furthermore, when the user is at home, the reception unit can preferentially display training purposes that can be performed at home. Thereby, the reception unit considers the user's geographical location information, enabling prioritized input of highly relevant training purposes. Part or all of the above-described processing in the reception unit may be performed using, for example, AI, or may be performed without using AI. Specifically, the present reception unit comprises a geofencing function that collates location information (latitude, longitude, altitude) acquired from GPS, Wi-Fi, Bluetooth beacon, etc. with a map database (POI: Point of Interest). Input to the reception unit is current coordinate data and position accuracy information. The system uses a spatial index such as R-Tree to search for and identify facilities (gym, park, public pool, home) around the current location at high speed. Based on the category of the identified place (e.g., “sports gym”), context-aware filtering logic operates. For example, when it is determined that the place is within a geofence of a “contracted gym”, the reception unit displays purposes requiring gym-specific equipment such as “machine muscle training” and “treadmill” at the top of the list. Conversely, when it is determined as “home”, purposes requiring no equipment such as “bodyweight training” and “yoga” are prioritized. Output is a purpose list weighted by the place context. This presents only executable options suitable for the user's physical environment, improving UI convenience.

The reception unit can analyze social media activity of the user when the training purpose is input, and propose a relevant purpose. The reception unit analyzes, for example, social media activity of the user when the training purpose is input, and proposes a relevant purpose. For example, the reception unit proposes a relevant training purpose based on training content shared by the user on social media. Also, the reception unit can propose a training purpose with reference to training content of a fitness influencer followed by the user. Furthermore, the reception unit can analyze a trend of a fitness community in which the user participates, and propose a relevant training purpose. Thereby, the reception unit analyzes the user's social media activity, enabling proposal of a relevant training purpose. Part or all of the above-described processing in the reception unit may be performed using, for example, AI, or may be performed without using AI. Specifically, the present reception unit comprises a social listening module that analyzes unstructured data (posted text, images, “like” history, follow list) acquired through an API of a social media platform. Input data is the user's SNS activity log. The reception unit extracts keywords (e.g., “marathon”, “competition”, “weight loss”) from posted text using natural language processing (NLP), and identifies activity content (e.g., scenery during running, image of protein) from posted images using image recognition (CV). Furthermore, using a Graph Neural Network (GNN), the reception unit analyzes trends (popular workout challenges, etc.) within influencers followed by the user or communities to which the user belongs, and generates a user interest vector. Similarity (cosine similarity, etc.) between this vector and a vector of a training purpose is calculated, and a purpose with high similarity is proposed as a “trend recommendation”. For example, if a specific abdominal exercise is popular among influencers, that exercise is presented as a purpose candidate in a push notification manner. This arouses the user's desire for training using social motivation.

The generation unit can estimate an emotion of the user, and adjust a generation method of the training menu based on the estimated emotion of the user. The generation unit estimates, for example, an emotion of the user, and adjusts a generation method of the training menu based on the estimated emotion of the user. For example, when the user is relaxed, the generation unit generates a training menu that proceeds at a leisurely pace. Also, when the user is in a hurry, the generation unit can generate a training menu that is effective in a short time. Furthermore, when the user is excited, the generation unit can generate a training menu with visually stimulating effects. Thereby, the generation unit adjusts the generation method of the training menu based on the user's emotion, enabling provision of a training menu optimal for the user. Estimation of the emotion is realized using an emotion estimation function using, for example, an emotion engine or generative AI. The generative AI is text generation AI (e.g., LLM) or multimodal generation AI, but is not limited to such examples. Specifically, the present generation unit uses a parametric menu generation algorithm or a reinforcement learning agent that receives an emotional state as a parameter. Input is an emotion label (e.g., Relaxed, Rushed, Excited) and its intensity value output from the emotion estimation unit, in addition to the user's basic profile. Inside the generation logic, a “generation template” or “reward function weight” corresponding to each emotional state is defined. For example, when the emotion is “Relaxed”, the generation unit performs parameter adjustment such as setting a long interval time, selecting BGM with low BPM (Beats Per Minute), and setting an instruction of motion speed to “slowly”. On the other hand, in the case of “Rushed”, the generation unit sets “total training time” as a strict constraint condition (e.g., within 15 minutes), and executes an optimization solver that generates a menu in a circuit training format with shortened break times. Output data is detailed instruction data including not only items, number of times, and number of sets, but also tempo, break time, and production effects (color tone of screen and tone of voice guide). This dynamically constructs a training experience perfectly adapted to the user's psychological and situational context.

The generation unit can refer to past training data of the user when generating the training menu, and generate an optimal menu. The generation unit refers to, for example, past training data of the user when generating the training menu, and generates an optimal menu. For example, the generation unit generates a menu according to progress based on training content performed by the user in the past. Also, the generation unit can select effective exercises from the user's past training data and generate a menu. Furthermore, the generation unit can analyze the user's past training history and propose an optimal training menu. Thereby, the generation unit refers to the user's past training data, enabling generation of an optimal training menu. Part or all of the above-described processing in the generation unit may be performed using, for example, generative AI, or may be performed without using generative AI. Specifically, the present generation unit utilizes a Memory Network or an LSTM-based sequence model that stores the user's training history (time series data). Input data is a sequence of training logs (performed items, load weight, number of times, number of sets, implementation date, subjective intensity RPE) for the past several months. The generation unit calculates an appropriate load for the next time based on the “Principle of Progressive Overload” from this history data. For example, if the user was able to lift 50 kg 10 times in the past 5 bench presses, the model predicts and generates a setting with slightly increased load, such as 52.5 kg or 50 kg 12 times, as the next load. Also, if there is a tendency for performance to decrease on the day after performing a specific item, adjustment such as lowering the frequency of that item is also performed. Furthermore, using collaborative filtering, patterns of other successful users (users who achieved goals) having similar histories are referred to, and new items that the current user has not yet performed but are expected to be effective are explored (Exploration) and incorporated into the menu. Output is a specific menu plan optimized based on scientific training theory and data-driven inference.

The generation unit can customize the menu based on a health condition or lifestyle habits of the user when generating the training menu. The generation unit customizes, for example, the menu based on a health condition or lifestyle habits of the user when generating the training menu. For example, when the user's health condition is not good, the generation unit generates a light training menu. Also, the generation unit can generate an achievable training menu based on the user's lifestyle habits. Furthermore, the generation unit can refer to the user's health data, and customize and provide an appropriate training menu. Thereby, the generation unit customizes the training menu based on the user's health condition or lifestyle habits, enabling provision of an achievable training menu. Part or all of the above-described processing in the generation unit may be performed using, for example, generative AI, or may be performed without using generative AI. Specifically, the present generation unit comprises a constrained optimization algorithm or a rule-based inference engine that incorporates vital data and life log data as constraint conditions. Input data is physiological indices such as Heart Rate Variability (HRV), resting heart rate, sleep score, and stress level. The generation unit calculates a “recovery score” from these indices. For example, if HRV is low and lack of sleep is detected, the recovery score becomes low, and the generation unit executes logic to put high-intensity anaerobic exercise on a prohibited list and generate low-intensity aerobic exercise or stretching instead. Also, referring to lifestyle habit data (work schedule, commuting time), if there is only 30 minutes of free time on weekdays, a high-density menu that fits within that time frame is generated. The output menu is adjusted to aim for the maximum effect within a range that does not exceed the user's physical capacity and time constraints. This prevents overtraining syndrome and supports formation of sustainable training habits.

The generation unit can estimate an emotion of the user, and determine a priority of the training menu based on the estimated emotion of the user. The generation unit estimates, for example, an emotion of the user, and determines a priority of the training menu based on the estimated emotion of the user. For example, when the user has high motivation, the generation unit preferentially displays a challenging training menu. Also, when the user is tired, the generation unit can preferentially display a relaxing training menu. Furthermore, when the user feels stress, the generation unit can preferentially display a training menu useful for stress relief. Thereby, the generation unit determines the priority of the training menu based on the user's emotion, enabling provision of a training menu optimal for the user. Estimation of the emotion is realized using an emotion estimation function using, for example, an emotion engine or generative AI. The generative AI is text generation AI (e.g., LLM) or multimodal generation AI, but is not limited to such examples. Specifically, the present generation unit determines a presentation order of menus using Multi-objective Optimization or a bandit algorithm. Input is a plurality of menu candidates (to which attribute scores such as “intensity”, “fun”, and “exhilaration” are assigned respectively) and the user's current emotion vector. The generation unit has a scoring function that dynamically changes an importance weight of each attribute according to the emotion vector. For example, when the emotion is “high motivation”, the weight of the “intensity” attribute is set high, and a total score of a high-intensity menu is calculated to be high. Conversely, when “stress” is high, the weights of “exhilaration” and “relaxation” attributes are increased. The generation unit sorts menu candidates based on the calculated total scores, and presents high-ranking ones as “today's recommended menu”. Also, by using a contextual bandit algorithm, feedback (reward) as to whether the presented menu was actually selected and completed by the user is used to continuously learn and improve matching accuracy between emotion and menu attributes.

The generation unit can consider geographical location information of the user when generating the training menu, and generate an optimal menu. The generation unit considers, for example, geographical location information of the user when generating the training menu, and generates an optimal menu. For example, when the user is near a gym, the generation unit generates a training menu that can be performed at the gym. Also, when the user is near a park, the generation unit can generate a training menu that can be performed at the park. Furthermore, when the user is at home, the generation unit can generate a training menu that can be performed at home. Thereby, the generation unit considers the user's geographical location information, enabling generation of an optimal training menu. Part or all of the above-described processing in the generation unit may be performed using, for example, generative AI, or may be performed without using generative AI. Specifically, the present generation unit links an environment recognition module based on location information with an available equipment database (Equipment DB) for each environment. Input is GPS coordinates and facility information. When the system identifies the current location as a “specific fitness gym”, it acquires a machine list (e.g., leg press machine, cable machine available) installed in that gym from the DB, and includes items using those machines in candidates for menu generation. On the other hand, when the current location is a “park”, the presence or absence of a horizontal bar or a bench is estimated from map data, and items such as chin-ups and step-ups are generated. In the case of “home”, registration information of equipment owned by the user (presence or absence of dumbbells, etc.) is referred to, and a menu possible only with owned equipment or body weight is generated. Output is an executable menu that makes maximum use of resources in that place. This allows the user to avoid a situation where “I went to the place but cannot do it because there is no equipment”.

The generation unit can analyze social media activity of the user when generating the training menu, and customize the menu. The generation unit analyzes, for example, social media activity of the user when generating the training menu, and customizes the menu. For example, the generation unit generates a relevant training menu based on training content shared by the user on social media. Also, the generation unit can generate a training menu with reference to training content of a fitness influencer followed by the user. Furthermore, the generation unit can analyze a trend of a fitness community in which the user participates, and generate a relevant training menu. Thereby, the generation unit analyzes the user's social media activity, enabling generation of a relevant training menu. Part or all of the above-described processing in the generation unit may be performed using, for example, generative AI, or may be performed without using generative AI. Specifically, the present generation unit comprises a trend analysis AI that analyzes text and images on SNS to extract a “training trend”. Input is the user's timeline and “liked” post data. The generation unit identifies hashtags (e.g., #30DayChallenge, #HIIT) in posted text using natural language processing (NLP) and motions in videos (e.g., burpee jump) using image recognition. If the user frequently reacts to “abs challenge” videos of a specific influencer, the generation unit generates a menu imitating items and set configurations recommended by that influencer, and proposes it as “menu done by Mr./Ms. XX”. Also, by preferentially incorporating items popular in the community, the user's sense of belonging and competitive spirit are stimulated. Output is a customized menu reflecting social trends.

The analysis unit can estimate an emotion of the user, and adjust an analysis method of the exercise form based on the estimated emotion of the user. For example, the analysis unit estimates the user's emotion and adjusts the analysis method of the exercise form based on the estimated user's emotion. For example, when the user is relaxed, the analysis unit performs a detailed form analysis. Also, when the user is in a hurry, the analysis unit can perform a concise form analysis. Furthermore, when the user is excited, the analysis unit can perform a form analysis with visually stimulating effects added. Thereby, the analysis unit can provide an optimal form analysis for the user by adjusting the analysis method of the exercise form based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this analysis unit has control logic that switches the granularity of the analysis algorithm and the mode of feedback generation (NLG) according to the emotional state. The input is camera video and an emotion label. When the emotion is “relaxed” or “high willingness to learn”, the analysis unit performs a detailed angle analysis of all joints, detects even minute deviations (e.g., the elbow angle is 5 degrees shallow), and generates feedback with detailed explanatory text and slow-motion video. On the other hand, when “in a hurry” or “irritated”, the analysis unit shifts to a simple mode that determines only major errors (e.g., squat is too shallow), and limits feedback to short sentences such as “Deeper!” or simple OK/NG signs. In the “excited” state, flashy particle effects and sound effects are synthesized at the moment the form matches, presenting the analysis result like a game. The output is a presentation format of the analysis result adapted to the user's receptivity.

The analysis unit can refer to past exercise data of the user when analyzing the exercise form, and improve accuracy of the analysis. For example, the analysis unit refers to the user's past exercise data during the analysis of the exercise form to improve the accuracy of the analysis. For example, the analysis unit analyzes the current form based on exercise forms performed by the user in the past. Also, the analysis unit can identify points for improvement of the form from the user's past exercise data. Furthermore, the analysis unit can analyze the user's past exercise history and propose an optimal form. Thereby, the analysis unit can improve the accuracy of the analysis by referring to the user's past exercise data. Part or all of the above-described processing in the analysis unit may be performed using, for example, AI, or may be performed without using AI. Specifically, this analysis unit uses a personalized model that has learned the user's individual movement characteristics (habits). The input is the current motion frame and the user's normal motion dataset accumulated in the past. The analysis unit adapts to the user-specific skeletal structure and range of motion limitations using transfer learning or fine-tuning while basing on a general ideal form model. For example, if the feature that “the right ankle is stiff” has been learned from past data, the analysis unit relaxes the judgment threshold for the angle of the right ankle and instead focuses the analysis on the movement of the hip joint. Also, it compares the past form and the current form in time series, and outputs a relative improvement evaluation such as “the back is straighter than one month ago”. This realizes a fair and highly accurate analysis based on individual physical characteristics, rather than a uniform standard.

The analysis unit can perform analysis based on a health condition or lifestyle habits of the user when analyzing the exercise form. For example, the analysis unit performs analysis based on the user's health condition or lifestyle habits during the analysis of the exercise form. For example, if the user's health condition is not good, the analysis unit performs a lighter form analysis. Also, the analysis unit can perform a feasible form analysis based on the user's lifestyle habits. Furthermore, the analysis unit can refer to the user's health data and perform an appropriate form analysis. Thereby, the analysis unit can provide a feasible form analysis by analyzing the exercise form based on the user's health condition and lifestyle habits. Part or all of the above-described processing in the analysis unit may be performed using, for example, AI, or may be performed without using AI. Specifically, this analysis unit performs a biomechanics simulation linked with vital data. The input is motion video as well as heart rate and fatigue level data. When the user's fatigue level is high, the analysis unit temporarily widens the tolerance for form disorder, or conversely, switches to a “safety priority mode” that issues a warning more strictly than usual against dangerous form collapse (e.g., arching of the back) because the risk of injury increases. Also, if there is health data indicating back pain, the load on the lower back (intervertebral disc compression force, etc.) is estimated, and if the load is about to exceed a threshold, an instruction to stop the movement is issued immediately. The output is a dynamic safety evaluation considering the context of the health condition.

The analysis unit can estimate an emotion of the user, and adjust a display method of an analysis result of the exercise form based on the estimated emotion of the user. For example, the analysis unit estimates the user's emotion and adjusts the display method of the analysis result of the exercise form based on the estimated user's emotion. For example, when the user is nervous, the analysis unit provides a simple and highly visible display method. Also, when the user is relaxed, the analysis unit can provide a display method including detailed information. Furthermore, when the user is in a hurry, the analysis unit can provide a display method focusing on key points. Thereby, the analysis unit can provide an optimal display method for the user by adjusting the display method of the analysis result of the exercise form based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this analysis unit controls a rendering engine that dynamically generates UI/UX. The input is analysis result data and an emotion label. When the emotion indicates “tension” or “confusion”, the analysis unit reduces the amount of information on the screen and displays the result intuitively using only large text and colors (green=OK, red=NG). On the other hand, in a “relaxed” or “analytical” state, specialized information such as graphs of joint angles, superimposed display of trajectories, and numerical data is displayed in a dashboard format. Also, text-to-speech (TTS) technology is used to switch the voice tone of the feedback to an “encouraging tone” or “calm instruction” according to the emotion. The output is audiovisual feedback optimized for the user's cognitive state.

The analysis unit can consider geographical location information of the user when analyzing the exercise form, and perform analysis. For example, the analysis unit performs analysis considering the user's geographical location information during the analysis of the exercise form. For example, when the user is exercising at a gym, the analysis unit performs a form analysis considering the equipment of the gym. Also, when the user is exercising in a park, the analysis unit can perform a form analysis considering the environment of the park. Furthermore, when the user is exercising at home, the analysis unit can perform a form analysis considering the space at home. Thereby, the analysis unit can provide an optimal form analysis by considering the user's geographical location information. Part or all of the above-described processing in the analysis unit may be performed using, for example, AI, or may be performed without using AI. Specifically, this analysis unit uses an environment-adaptive vision model that combines background recognition and object detection. The input is video and location information. When the location is a “gym”, the analysis unit detects a training machine (e.g., lat pulldown machine) in the video and performs a form analysis along the movable trajectory of the machine. The correct way to sit and grip position specific to the machine are also subject to checking. In the case of “home”, it recognizes furniture and walls in the background, and activates a complementation algorithm that can continue estimation even if limbs go out of the screen, considering that movement is restricted in a narrow space. Also, it estimates the installation position of the camera (placed on the floor, placed on a table) and performs 3D transformation processing to correct distortion due to the angle. The output is an analysis result in which noise due to environmental factors is removed and evaluation is performed based on criteria suitable for the situation.

The analysis unit can analyze social media activity of the user when analyzing the exercise form, and propose points for improvement of the form. For example, the analysis unit analyzes the user's social media activity during the analysis of the exercise form and proposes points for improvement. For example, the analysis unit proposes points for improvement based on an exercise form shared by the user on social media. Also, the analysis unit can propose points for improvement with reference to a form of a fitness influencer followed by the user. Furthermore, the analysis unit can analyze trends of a fitness community in which the user participates, and propose points for improvement of the form. Thereby, the analysis unit can propose relevant points for improvement of the form by analyzing the user's social media activity. Part or all of the above-described processing in the analysis unit may be performed using, for example, AI, or may be performed without using AI. Specifically, this analysis unit analyzes video content on SNS and uses it as a comparison target. The input is the user's form video and an influencer's video acquired from SNS. The analysis unit extracts skeletal movements from both videos and performs time-series matching. Then, it generates a comparative improvement proposal such as “Your squat has a squatting speed that is too fast compared to athlete XX whom you follow.” Also, a video shared as a “correct form” within the community is set as a reference (correct data), and deviation from it is pointed out. The output is highly convincing improvement advice based on a target the user admires or a group to which the user belongs.

The proposal unit can estimate an emotion of the user, and adjust a proposal method of the meal plan based on the estimated emotion of the user. For example, the proposal unit estimates the user's emotion and adjusts the proposal method of the meal plan based on the estimated user's emotion. For example, when the user is relaxed, the proposal unit proposes a meal plan that proceeds at a leisurely pace. Also, when the user is in a hurry, the proposal unit can propose a meal plan that can be prepared in a short time. Furthermore, when the user is excited, the proposal unit can propose a meal plan with visually stimulating effects added. Thereby, the proposal unit can provide an optimal meal plan for the user by adjusting the proposal method of the meal plan based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this proposal unit has recipe search/generation logic according to the emotional context. The input is emotion data and nutritional requirements. When the emotion is “stress” or “fatigue”, the proposal unit applies filtering to minimize “cooking time” and proposes a combination purchasable at a convenience store or a simple recipe that does not use a kitchen knife. Also, ingredients containing nutrients (Vitamin C, Calcium, etc.) effective for stress reduction are prioritized. On the other hand, in the case of “relaxed” or “fun” emotions, it proposes elaborate dishes where the cooking process can be enjoyed or dishes that look beautiful. The output is a highly feasible meal plan considering the user's mental resources.

The proposal unit can refer to past meal data of the user when proposing the meal plan, and propose an optimal plan. For example, the proposal unit refers to the user's past meal data during the proposal of the meal plan to propose an optimal plan. For example, the proposal unit proposes a meal plan according to progress based on meal contents consumed by the user in the past. Also, the proposal unit can select effective meal contents from the user's past meal data and propose a plan. Furthermore, the proposal unit can analyze the user's past meal history and propose an optimal meal plan. Thereby, the proposal unit can propose an optimal meal plan by referring to the user's past meal data. Part or all of the above-described processing in the proposal unit may be performed using, for example, AI, or may be performed without using AI. Specifically, this proposal unit uses a preference model that has learned the user's food log. The input is past meal images and text records. The proposal unit analyzes ingredients the user eats frequently, ingredients the user avoids, and meal time patterns. For example, if many recipes using “chicken breast” have been adopted in the past, a new variation (change in seasoning, etc.) using chicken breast is proposed. Also, a menu that was proposed in the past but rejected (not eaten) is learned as negative feedback and excluded from the next proposal. Furthermore, weight change data and meal data are matched to identify a “meal pattern with high weight loss effect” for that user, and a plan to reproduce it is generated. The output is a highly accurate recommendation list based on the user's preferences and track record.

The proposal unit can customize the plan based on a health condition or lifestyle habits of the user when proposing the meal plan. For example, the proposal unit customizes the plan based on the user's health condition or lifestyle habits during the proposal of the meal plan. For example, if the user's health condition is not good, the proposal unit proposes a lighter meal plan. Also, the proposal unit can propose a feasible meal plan based on the user's lifestyle habits. Furthermore, the proposal unit can refer to the user's health data and customize and provide an appropriate meal plan. Thereby, the proposal unit can provide a feasible meal plan by customizing the meal plan based on the user's health condition and lifestyle habits. Part or all of the above-described processing in the proposal unit may be performed using, for example, AI, or may be performed without using AI. Specifically, this proposal unit is equipped with an inference engine that cooperates with a medical/nutritional knowledge graph. The input is vital data, allergy information, and life rhythm. If there is data that the user tends to have high blood pressure, the proposal unit filters and proposes only recipes with strictly limited salt content. Also, if the lifestyle habit involves many “late returns home at night”, a late-night snack menu that is easy to digest and does not disturb sleep is proposed. Furthermore, considering biorhythms such as a woman's menstrual cycle, a menu that supplements iron and magnesium, which tend to be deficient at specific times, is automatically incorporated. The output is a personalized nutrition management plan perfectly adapted to the individual's constitution and living environment.

The proposal unit can estimate an emotion of the user, and determine a priority of the meal plan based on the estimated emotion of the user. For example, the proposal unit estimates the user's emotion and determines the priority of the meal plan based on the estimated user's emotion. For example, when the user is highly motivated, the proposal unit preferentially displays a challenging meal plan. Also, when the user is tired, the proposal unit can preferentially display a relaxing meal plan. Furthermore, when the user feels stressed, the proposal unit can preferentially display a meal plan useful for stress relief. Thereby, the proposal unit can provide an optimal meal plan for the user by determining the priority of the meal plan based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this proposal unit uses a contextual bandit algorithm that uses an emotional state as a context for reward prediction. The input is a plurality of meal plan candidates and an emotion vector. The system predicts which plan (e.g., strict carbohydrate restriction) will maximize the probability that the user will accept (click) and execute it in the current emotional state (e.g., high motivation). When motivation is high, a “result-oriented” plan is ranked higher, and when tired, “convenience-oriented” or “comfort food” is ranked higher. The output is a display order of plans optimized based on the user's acceptance probability.

The proposal unit can consider geographical location information of the user when proposing the meal plan, and propose an optimal plan. For example, the proposal unit proposes an optimal plan considering the user's geographical location information during the proposal of the meal plan. For example, when the user is near a supermarket, the proposal unit proposes a meal plan using ingredients purchasable at the supermarket. Also, when the user is near a restaurant, the proposal unit can propose a meal plan referring to a menu provided at the restaurant. Furthermore, when the user is at home, the proposal unit can propose a meal plan that can be easily prepared at home. Thereby, the proposal unit can propose an optimal meal plan by considering the user's geographical location information. Part or all of the above-described processing in the proposal unit may be performed using, for example, AI, or may be performed without using AI. Specifically, this proposal unit is equipped with an O2O (Online to Offline) module that links location information with an external store inventory API or restaurant menu DB. The input is GPS coordinates. When the user is near a specific supermarket, the proposal unit refers to the special sale information and inventory data of that store and proposes “a recipe using chicken that can be bought cheaply now”. When in a dining-out area, a specific menu (e.g., “Grilled fish set meal at XX cafeteria, less rice”) matching the user's nutritional goal (e.g., low fat, high protein) is recommended from menus of nearby restaurants. The output is an immediately executable proposal based on physically accessible ingredients or dishes.

The proposal unit can analyze social media activity of the user when proposing the meal plan, and customize the plan. For example, the proposal unit analyzes the user's social media activity during the proposal of the meal plan and customizes the plan. For example, the proposal unit proposes a relevant meal plan based on meal contents shared by the user on social media. Also, the proposal unit can propose a meal plan with reference to meal contents of a fitness influencer followed by the user. Furthermore, the proposal unit can analyze trends of a fitness community in which the user participates, and propose a relevant meal plan. Thereby, the proposal unit can propose a relevant meal plan by analyzing the user's social media activity. Part or all of the above-described processing in the proposal unit may be performed using, for example, AI, or may be performed without using AI. Specifically, this proposal unit is equipped with an image recognition AI (FoodAI) that analyzes food images on SNS. The input is SNS post data. If the user frequently “likes” images of “avocado toast”, the system generates and proposes a healthy recipe using avocado. Also, it analyzes meal contents published by an influencer by text mining, calculates the nutritional value thereof, and then proposes an “influencer-style menu” in which the amount is adjusted according to the user's goal. The output is an attractive meal plan reflecting the user's potential food interests and trends.

The management unit can estimate an emotion of the user, and adjust a method of progress management based on the estimated emotion of the user. For example, the management unit estimates the user's emotion and adjusts the method of progress management based on the estimated user's emotion. For example, when the user is relaxed, the management unit performs progress management that proceeds at a leisurely pace. Also, when the user is in a hurry, the management unit can provide a management method allowing quick confirmation of progress. Furthermore, when the user is excited, the management unit can perform progress management with visually stimulating effects added. Thereby, the management unit can provide optimal progress management for the user by adjusting the method of progress management based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this management unit is equipped with a reinforcement learning agent that optimizes notification timing and message content. The input is an emotional state and past reaction history. When the user is “relaxed”, detailed progress information such as a weekly report is notified to encourage deep reflection. On the other hand, when in a “busy” or “stress” state, the notification is suspended, or only a short message understandable at a glance such as “1 kg more to the goal!” is sent. Also, in the display of the progress graph, the granularity of information is adjusted, such as displaying a detailed line graph when the emotion is good, and displaying a simple achievement rate bar when the emotion is bad. The output is a management interface that maximizes engagement while minimizing the user's emotional burden.

The management unit can refer to past training data of the user during progress management, and improve accuracy of the management. For example, the management unit refers to the user's past training data during progress management to improve the accuracy of the management. For example, the management unit manages progress based on training contents performed by the user in the past. Also, the management unit can propose an effective progress management method from the user's past training data. Furthermore, the management unit can analyze the user's past training history and provide an optimal progress management method. Thereby, the management unit can improve the accuracy of the management by referring to the user's past training data. Part or all of the above-described processing in the management unit may be performed using, for example, AI, or may be performed without using AI. Specifically, this management unit uses a regression analysis or Bayesian estimation model that models a growth curve. The input is time-series data of past training load and physical changes. The management unit learns the user's individual “responsiveness of results to effort” and predicts future progress. For example, it makes a prediction such as “Goal achievement in 3 weeks if this pace continues”, and displays the prediction line (with confidence interval) on a graph. Also, it detects a pattern of frustration in the past (e.g., quitting after 2 weeks if going to the gym more than 4 times a week), and issues a warning in advance to encourage slowing down if a similar pattern is likely to occur. The output is highly accurate budget-actual management information backed by data.

The management unit can perform management based on a health condition or lifestyle habits of the user during progress management. For example, the management unit performs management based on the user's health condition or lifestyle habits during progress management. For example, if the user's health condition is not good, the management unit performs lighter progress management. Also, the management unit can perform feasible progress management based on the user's lifestyle habits. Furthermore, the management unit can refer to the user's health data and perform appropriate progress management. Thereby, the management unit can provide feasible progress management by performing progress management based on the user's health condition and lifestyle habits. Part or all of the above-described processing in the management unit may be performed using, for example, AI, or may be performed without using AI. Specifically, this management unit is equipped with a risk management module that evaluates health risks. The input is vital data and life logs. If the user's lack of sleep or high stress state continues, the management unit automatically extends the “goal achievement deadline” and performs a dynamic schedule change to downwardly revise the daily quota. This is to prevent health damage due to unreasonable progress management. Also, during the menstrual cycle or poor physical condition, logic is applied such that weight gain is processed as “swelling” and is not treated as a regression of progress (so as not to lower the user's motivation). The output is a flexible progress management plan that prioritizes physical and mental health.

The management unit can estimate an emotion of the user, and determine a priority of progress management based on the estimated emotion of the user. For example, the management unit estimates the user's emotion and determines the priority of progress management based on the estimated user's emotion. For example, when the user is highly motivated, the management unit preferentially performs challenging progress management. Also, when the user is tired, the management unit can preferentially perform relaxing progress management. Furthermore, when the user feels stressed, the management unit can preferentially perform progress management useful for stress relief. Thereby, the management unit can provide optimal progress management for the user by determining the priority of progress management based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this management unit introduces an emotion variable into a task scheduling algorithm. The input is unfinished training tasks or measurement tasks and an emotion vector. When motivation is high, high-load and challenging tasks such as “maximum weight measurement (1RM test)” and “time trial” are preferentially scheduled and notified. Conversely, when tired, low-load tasks such as “confirmation of stretching implementation” or “weight recording only” are prioritized. The output is a ToDo list dynamically reordered according to the user's psychological receptivity.

The management unit can consider geographical location information of the user during progress management, and perform management. For example, the management unit performs management considering the user's geographical location information during progress management. For example, when the user is near a gym, the management unit performs progress management that can be done at the gym. Also, when the user is near a park, the management unit can perform progress management that can be done at the park. Furthermore, when the user is at home, the management unit can perform progress management that can be done at home. Thereby, the management unit can provide optimal progress management by considering the user's geographical location information. Part or all of the above-described processing in the management unit may be performed using, for example, AI, or may be performed without using AI. Specifically, this management unit has a task management function using a location information trigger. The input is GPS coordinates. When it detects that the user has arrived at the gym, the management unit automatically performs “check-in” processing, displays the training menu for the day, and pops up a result input screen after completion. When in a park, a running distance measurement mode is proposed. When it detects that the user has returned home, a reminder for “weight measurement” is sent. The output is a timely and hassle-free progress recording interface linked to the context of location.

The management unit can analyze social media activity of the user during progress management, and customize a method of the management. For example, the management unit analyzes the user's social media activity during progress management and customizes the method of the management. For example, the management unit proposes a relevant progress management method based on progress contents shared by the user on social media. Also, the management unit can propose a management method with reference to a progress management method of a fitness influencer followed by the user. Furthermore, the management unit can analyze trends of a fitness community in which the user participates, and propose a relevant progress management method. Thereby, the management unit can propose a relevant progress management method by analyzing the user's social media activity. Part or all of the above-described processing in the management unit may be performed using, for example, AI, or may be performed without using AI. Specifically, this management unit performs analysis applying social comparison theory. The input is SNS data. If the user is a type who likes to share results on SNS (extrinsic motivation), the management unit automatically generates progress data in a “shareable image format (graph with stamps, etc.)” and encourages posting. Also, if competing with a friend, a progress comparison graph with the friend is displayed. If an influencer adopts a method of “uploading abdominal muscle images every day”, a “daily body shape photo log” function is proposed imitating it. The output is a management method that increases the continuation rate using social connections.

The image generation unit can estimate an emotion of the user, and adjust a method of image generation based on the estimated emotion of the user. For example, the image generation unit estimates the user's emotion and adjusts the method of image generation based on the estimated user's emotion. For example, when the user is relaxed, the image generation unit generates an image that proceeds at a leisurely pace. Also, when the user is in a hurry, the image generation unit can provide an image that can be generated in a short time. Furthermore, when the user is excited, the image generation unit can generate an image with visually stimulating effects added. Thereby, the image generation unit can provide an optimal image for the user by adjusting the method of image generation based on the user's emotion. The estimation of the emotion is realized using an emotion estimation function using, for example, an emotion engine or generative AI. The generative AI is a text generation AI (e.g., LLM) or a multimodal generative AI, but is not limited to such examples. Specifically, this image generation unit controls style parameters and rendering settings of a generative model (GAN or Diffusion) according to the emotion. The input is an emotion vector. When the emotion is “excitement” or “elation”, the image generation unit increases saturation, strengthens contrast, and generates a “hero-style” before-after image synthesizing energetic effects (rays, flames, etc.) in the background. On the other hand, when “anxious” or “depressed”, it uses soft lighting and calm color tones to generate a natural image that does not overemphasize excessive changes, giving a sense of security. Also, the speed of the generation process itself is adjusted, and when in a hurry, a high-speed preview at low resolution is prioritized. The output is image content that is close to the user's emotion and maximizes the psychological effect.

The image generation unit can refer to past training data of the user during image generation, and improve accuracy of the generation. For example, the image generation unit refers to the user's past training data during image generation to improve the accuracy of the generation. For example, the image generation unit generates an image according to progress based on training contents performed by the user in the past. Also, the image generation unit can select an effective image from the user's past training data and generate it. Furthermore, the image generation unit can analyze the user's past training history and generate an optimal image. Thereby, the image generation unit can improve the accuracy of the generation by referring to the user's past training data. Part or all of the above-described processing in the image generation unit may be performed using, for example, generative AI, or may be performed without using generative AI. Specifically, this image generation unit includes a physical transformation simulator that has learned the correlation between training load and muscle development. The input is a history of past training items and loads. If the user is focusing on “bench press”, the image generation unit performs local image deformation processing (warping) that selectively hypertrophies the regions of the pectoralis major and triceps brachii, rather than changing the whole body uniformly. Thereby, it generates a realistic prediction image conforming to the training content, such as “which part changes and how”, not just “lost weight” or “gained weight”. The output is a convincing future prediction diagram reflecting the individual's points of effort.

The image generation unit can customize an image based on a health condition or lifestyle habits of a user when generating the image. For example, the image generation unit customizes the image based on the health condition or lifestyle habits of the user when generating the image. For example, if the health condition of the user is not good, the image generation unit generates a moderate image. Also, the image generation unit can generate an achievable image based on the lifestyle habits of the user. Furthermore, the image generation unit can refer to health data of the user and customize and provide an appropriate image. Thereby, the image generation unit can provide an achievable image by customizing the image based on the health condition or lifestyle habits of the user. Part or all of the above-described processing in the image generation unit may be performed using, for example, a generative AI, or may be performed without using a generative AI. Specifically, the present image generation unit has constraint logic for performing image generation within a medically reasonable range. Inputs are age, gender, and medical examination data. For example, considering the age and hormone balance of the user, generation of an image with a muscle mass or body fat percentage (e.g., 3% body fat percentage) that is realistically unachievable is restricted. Instead, an image representing a “best condition” for the user, which is healthy and toned, is generated. Also, if there is poor posture (such as stoop) as health data, an image in a state where not only muscles are gained but also posture is improved is generated to visualize merits in terms of health. The output is not a fantasy but an achievable goal image based on medical evidence.

The image generation unit can estimate an emotion of the user and adjust a display method of the generated image based on the estimated emotion of the user. For example, the image generation unit estimates the emotion of the user and adjusts the display method of the generated image based on the estimated emotion of the user. For example, if the user is nervous, the image generation unit provides a simple and highly visible display method. Also, if the user is relaxed, the image generation unit can provide a display method including detailed information. Furthermore, if the user is in a hurry, the image generation unit can provide a display method focusing on main points. Thereby, the image generation unit can provide an optimal display method for the user by adjusting the display method of the generated image based on the emotion of the user. The estimation of the emotion is realized using an emotion estimation function using, for example, an emotion engine or a generative AI. The generative AI is a text generation AI (e.g., LLM) or a multimodal generative AI, but is not limited to such examples. Specifically, the present image generation unit controls a display module including AR (Augmented Reality) or a 3D model viewer. Inputs are the generated image and emotion data. If the user is “curious” and relaxed, an interactive viewer capable of rotating the generated 3D body model 360 degrees or zooming is displayed to allow checking details such as muscle definition. On the other hand, if the user is “unconfident” or “nervous”, since a realistic image that is too detailed may be counterproductive, a soft image processed in an illustration style or a silhouette style is displayed, or a simple comparison image emphasizing only parts with large changes is displayed. The output is a presentation format considering psychological receptivity of the user.

The image generation unit can generate an image in consideration of geographical location information of the user when generating the image. For example, the image generation unit generates the image in consideration of the geographical location information of the user when generating the image. For example, if the user is near a gym, the image generation unit generates an image reflecting training content that can be performed at the gym. Also, if the user is near a park, the image generation unit can generate an image reflecting training content that can be performed at the park. Furthermore, if the user is at home, the image generation unit can generate an image reflecting training content that can be performed at home. Thereby, the image generation unit can generate an optimal image by considering the geographical location information of the user. Part or all of the above-described processing in the image generation unit may be performed using, for example, a generative AI, or may be performed without using a generative AI. Specifically, the present image generation unit uses a background synthesis (inpainting) technology. Inputs are a user image and location information. If the user is at a “seaside park”, a background of a generated image of the future self is synthesized with scenery of the park or a beach to allow the user to imagine “self exercising with an ideal body shape there”. If the user is at a gym, a background as if posing in front of a mirror at the gym is synthesized. Thereby, the user can easily link the current environment with the appearance after achieving the goal, enabling realistic image training. The output is a context-aware synthesized image.

The image generation unit can analyze social media activity of the user and customize an image when generating the image. For example, the image generation unit analyzes the social media activity of the user and customizes the image when generating the image. For example, the image generation unit generates a relevant image based on training content shared by the user on social media. Also, the image generation unit can generate an image with reference to training content of a fitness influencer followed by the user. Furthermore, the image generation unit can analyze trends of a fitness community in which the user participates and generate a relevant image. Thereby, the image generation unit can generate a relevant image by analyzing the social media activity of the user. Part or all of the above-described processing in the image generation unit may be performed using, for example, a generative AI, or may be performed without using a generative AI. Specifically, the present image generation unit uses a style transfer model that has learned an art style or composition that looks good on SNS (Instagrammable). The input is trend information of SNS. If a “monochrome muscle photo” is popular on SNS currently, the generated image is processed with a monochrome filter and output as a dramatic image with emphasized shading. Also, a pose frequently taken by an influencer is extracted by skeleton estimation, and the generated image of the user is deformed (rigging & posing) into that pose and generated. The output is high-quality image content satisfying a desire for approval that the user wants to post to SNS as it is.

The system according to the embodiment is not limited to the above-described examples, and various modifications are possible, for example, as follows. Specifically, the present system can be implemented on various hardware and network architectures, such as not only a centralized cloud server configuration but also an edge AI configuration in which part or all of inference processing is performed on an edge device (smartphone, wearable terminal) side, or a configuration in which health data and training history of the user are decentrally managed using blockchain technology to enhance privacy and security. Also, each functional unit (reception, generation, analysis, etc.) may be configured not by a single model but by a plurality of specialized models (ensemble learning), and a configuration in which a model is updated without transmitting privacy data of the user to a server using federated learning may be adopted.

The reception unit can analyze a past training history of the user and propose an input method of a training purpose. For example, the reception unit automatically displays, as candidates, training purposes frequently input by the user in the past. Also, the reception unit can preferentially propose an input method (voice, text, etc.) used by the user in the past. Furthermore, the reception unit can predict and propose a training purpose to be used in a specific time zone from the past training history of the user. Thereby, the reception unit can propose an optimal purpose input method by analyzing the past training history of the user. Specifically, the reception unit in this modification functions as a personalized UI agent that learns an input pattern for each user. Using input history data (timestamp, input content, input modality) as teacher data, a lightweight machine learning model such as a decision tree or random forest is trained within a user terminal. Thereby, a local rule such as “probability of selecting ‘yoga’ by voice input is high on Friday night” is extracted, and when an application is started at the corresponding date and time, a prompt “Do you want to start yoga?” is automatically displayed in a voice input standby state. This minimizes the trouble of input to the limit.

The generation unit can estimate an emotion of the user and adjust a generation method of a training menu based on the estimated emotion of the user. For example, if the user is relaxed, the generation unit generates a training menu that proceeds at a leisurely pace. Also, if the user is in a hurry, the generation unit can generate a training menu that is effective in a short time. Furthermore, if the user is excited, the generation unit can generate a training menu to which visually stimulating effects are added. Thereby, the generation unit can provide an optimal training menu for the user by adjusting the generation method of the training menu based on the emotion of the user. Specifically, the generation unit in this modification generates training session data to which a result of emotion recognition is added as metadata. A menu generation algorithm applies a “modifier” according to an emotion parameter to a basic exercise program. For example, if the emotion is “anger”, aerobic exercise including divergent actions such as punching and kicking is dynamically inserted, and modification to increase the tempo of BGM is performed. Thereby, the training is made to function not only as mere physical exercise but also as a means of emotion regulation.

The analysis unit can perform analysis of an exercise form based on a health condition or lifestyle habits of the user. For example, if the health condition of the user is not good, the analysis unit performs moderate form analysis. Also, the analysis unit can perform achievable form analysis based on the lifestyle habits of the user. Furthermore, the analysis unit can refer to health data of the user and perform appropriate form analysis. Thereby, the analysis unit can provide achievable form analysis by analyzing the exercise form based on the health condition or lifestyle habits of the user. Specifically, the analysis unit in this modification strengthens API cooperation with medical healthcare data and incorporates orthopedic risk assessment. If the user has a medical history of “right anterior cruciate ligament injury”, the analysis unit intensively calculates a load vector applied to the right knee, and issues a warning with a stricter criterion than usual for an action in which the knee goes inward (knee-in). Also, an adaptive analysis criterion (adaptive threshold) that gradually expands an allowable range of a range of motion in accordance with progress of rehabilitation is adopted.

The proposal unit can estimate an emotion of the user and adjust a proposal method of a meal plan based on the estimated emotion of the user. For example, if the user is relaxed, the proposal unit proposes a meal plan that proceeds at a leisurely pace. Also, if the user is in a hurry, the proposal unit can propose a meal plan that can be prepared in a short time. Furthermore, if the user is excited, the proposal unit can propose a meal plan to which visually stimulating effects are added. Thereby, the proposal unit can provide an optimal meal plan for the user by adjusting the proposal method of the meal plan based on the emotion of the user. Specifically, the proposal unit in this modification applies Nudge theory based on eating behavior psychology. If it is estimated that the emotion is unstable and a risk of overeating (emotional eating) is high, the proposal unit dynamically generates a UI design that induces the user to make a healthy choice unconsciously by displaying an image of high-calorie food in a small size or displaying a healthy alternative food (e.g., fruit) in a large size with an attractive image.

The management unit can improve accuracy of progress management by referring to past training data of the user. For example, the management unit manages progress based on training content performed by the user in the past. Also, the management unit can propose an effective progress management method from the past training data of the user. Furthermore, the management unit can analyze a past training history of the user and provide an optimal progress management method. Thereby, the management unit can improve the accuracy of management by referring to the past training data of the user. Specifically, the management unit in this modification uses cohort analysis. The user is classified into a cluster such as “short-term intensive type”, “steady type”, or “weekend type” based on past data. Then, referring to a success case (best practice) of another user belonging to the same cluster, a management schedule with the highest success probability for the user (e.g., progress check three times a week is optimal) is applied. This constructs a reasonable management system suitable for individual characteristics.

The image generation unit can estimate an emotion of the user and adjust a display method of the generated image based on the estimated emotion of the user. For example, if the user is nervous, the image generation unit provides a simple and highly visible display method. Also, if the user is relaxed, the image generation unit can provide a display method including detailed information. Furthermore, if the user is in a hurry, the image generation unit can provide a display method focusing on main points. Thereby, the image generation unit can provide an optimal display method for the user by adjusting the display method of the generated image based on the emotion of the user. Specifically, the image generation unit in this modification controls presentation timing and production of the generated image. If it is estimated that the user is depressed (negative emotion), an image emphasizing that “I have grown this much” by comparing the past self and the current self is displayed with animation along with an encouraging message. Thereby, the image generation function is utilized not as a mere simulation but as a mental support tool.

The reception unit can adjust an input method of a training purpose in consideration of geographical location information of the user. For example, if the user is near a gym, the reception unit preferentially displays a training purpose that can be performed at the gym. Also, if the user is near a park, the reception unit can preferentially display a training purpose that can be performed at the park. Furthermore, if the user is at home, the reception unit can preferentially display a training purpose that can be performed at home. Thereby, the reception unit can allow the user to preferentially input a highly relevant training purpose by considering the geographical location information of the user. Specifically, the reception unit in this modification automatically learns a living sphere (home, workplace, gym) of the user from location information history and creates a “context profile” for each place. For example, when the user is at the “workplace”, “short-time stretch” or “exercise to relieve eye strain” is preferentially displayed, and when the user is at the “gym”, “muscle training” is preferentially displayed. This saves the user the trouble of searching for a purpose each time and enables the user to start an optimal action immediately on the spot.

The generation unit can analyze social media activity of the user and customize a training menu. For example, the generation unit generates a relevant training menu based on training content shared by the user on social media. Also, the generation unit can generate a training menu with reference to training content of a fitness influencer followed by the user. Furthermore, the generation unit can analyze trends of a fitness community in which the user participates and generate a relevant training menu. Thereby, the generation unit can generate a relevant training menu by analyzing the social media activity of the user. Specifically, the generation unit in this modification has a function of analyzing information of a “challenge project (e.g., 30-day plank challenge)” acquired through an API of SNS, and automatically converting and importing the challenge content as a training menu in the system. Thereby, the user can implement trending training found on SNS under the management of the present system while receiving correct form analysis and progress management.

The analysis unit can estimate an emotion of the user and adjust a display method of an analysis result of an exercise form based on the estimated emotion of the user. For example, if the user is nervous, the analysis unit provides a simple and highly visible display method. Also, if the user is relaxed, the analysis unit can provide a display method including detailed information. Furthermore, if the user is in a hurry, the analysis unit can provide a display method focusing on main points. Thereby, the analysis unit can provide an optimal display method for the user by adjusting the display method of the analysis result of the exercise form based on the emotion of the user. Specifically, the analysis unit in this modification adjusts “strictness” of feedback according to the emotion. When the user loses confidence, the analysis unit operates in a “positive feedback mode” in which the analysis unit overlooks some disorder of the form and praises a part that was done well. Conversely, when the user is overconfident, the analysis unit operates in a “strict mode” in which even a small mistake is pointed out, promoting injury prevention and skill improvement.

The proposal unit can customize a meal plan based on a health condition or lifestyle habits of the user. For example, if the health condition of the user is not good, the proposal unit proposes a moderate meal plan. Also, the proposal unit can propose an achievable meal plan based on the lifestyle habits of the user. Furthermore, the proposal unit can refer to health data of the user and customize and provide an appropriate meal plan. Thereby, the proposal unit can provide an achievable meal plan by customizing the meal plan based on the health condition or lifestyle habits of the user. Specifically, the proposal unit in this modification cooperates with a smart refrigerator or purchase history of an EC site. Ingredients in the refrigerator of the user (inventory data) and the health condition (necessary nutrients) are matched to propose a “recipe that can be made with currently available ingredients and is good for health”. This realizes realistic and easy-to-execute meal management while reducing food loss.

A flow of processing of Example of the Embodiment will be briefly described below. Specifically, a series of processing processes in the present system is controlled as a state machine designed based on an Event-Driven Architecture. Each step is executed asynchronously triggered by a completion event of a previous step, and data consistency and processing efficiency are achieved.

Step 1: The reception unit receives an input of a training purpose from the user. For example, the user inputs a purpose such as dieting or muscle gain. Step 2: The generation unit generates a personal training menu based on the purpose received by the reception unit. For example, the generation unit generates an exercise menu according to the user's purpose. For example, for a user whose purpose is muscle gain, the generation unit generates a menu for muscle training. Step 3: The analysis unit analyzes an exercise form based on the menu generated by the generation unit. For example, the analysis unit analyzes the form when the user performs an exercise, and checks whether the exercise is being performed with an appropriate form. Step 4: The proposal unit proposes a meal plan based on the menu generated by the generation unit. For example, the proposal unit generates a meal plan according to the user's purpose and provides the meal plan to the user. Step 5: The management unit performs progress management based on the menu generated by the generation unit. For example, the management unit manages progress of the user's training and provides support for achieving a goal. Step 6: The image generation unit presents an appearance of the user before and after achieving the purpose using an image generation function. For example, the image generation unit generates images of the appearance before and after the user starts the training, and presents the images to the user. Thereby, the user can increase motivation for achieving the goal. Specifically, in Step 1,, a natural language processing unit analyzes input text of the user, extracts an intent vector, and stores the intent vector in a database. In subsequent Step 2, a recommendation engine refers to the intent vector and a user profile, and generates a menu list using an optimization algorithm. In Step 3, when the user executes the menu, a computer vision module performs skeleton tracking in real time, calculates a deviation of the form, and provides immediate feedback. In parallel, in Step 4, a nutrition management module recalculates and presents a meal plan considering calories burned by the exercise. In Step 5, these activity logs are recorded in a time-series database, and a progress prediction model updates a goal achievement rate. Finally, in Step 6, based on updated progress data, an image generation AI renders a future body image and displays the image on a dashboard of the user, thereby completing a feedback loop.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described reception unit, generation unit, analysis unit, proposal unit, management unit, and image generation unit is implemented by, for example, at least one of the smart device 14 and the data processing apparatus 12. For example, the reception unit is implemented by a control unit 46A of the smart device 14, and a user inputs a training purpose. The generation unit is implemented by, for example, a specific processing unit 290 of the data processing apparatus 12, and generates a personal training menu according to the user's purpose. The analysis unit is implemented by, for example, the control unit 46A of the smart device 14, and analyzes an exercise form. The proposal unit is implemented by, for example, the specific processing unit 290 of the data processing apparatus 12, and proposes a meal plan. The management unit is implemented by, for example, the specific processing unit 290 of the data processing apparatus 12, and performs progress management. The image generation unit is implemented by, for example, the control unit 46A of the smart device 14, and presents “one's own appearance” before and after achieving the purpose using an image generation function. The correspondence between each unit and the device or the control unit is not limited to the above-described example, and various modifications are possible.

### Second Embodiment

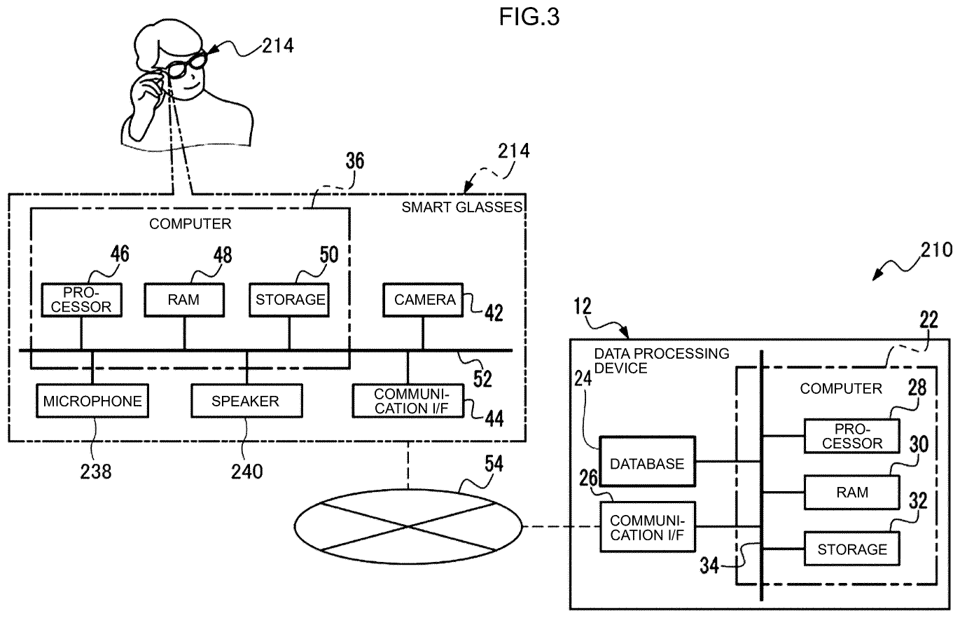

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

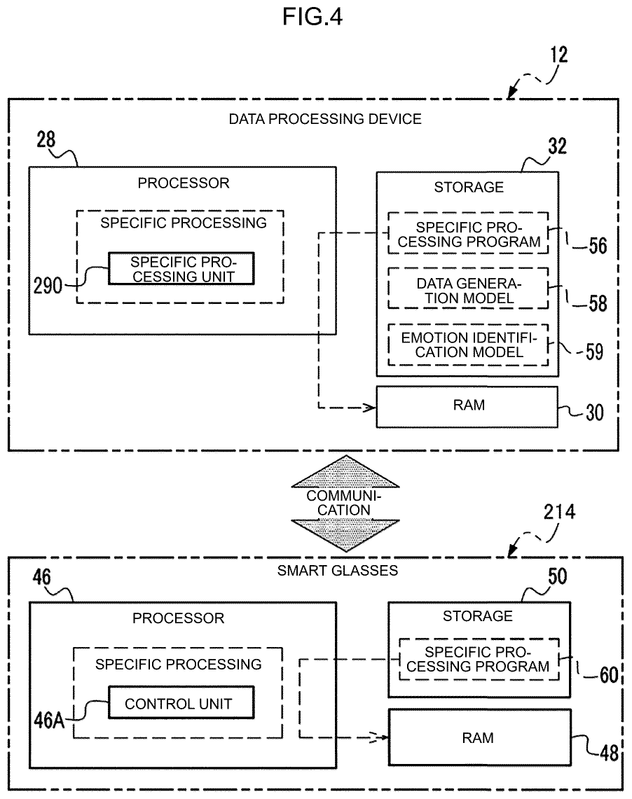

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described reception unit, generation unit, analysis unit, proposal unit, management unit, and image generation unit is implemented by, for example, at least one of the smart glasses 214 and the data processing apparatus 12. For example, the reception unit is implemented by a control unit 46A of the smart glasses 214, and a user inputs a training purpose. The generation unit is implemented by, for example, a specific processing unit 290 of the data processing apparatus 12, and generates a personal training menu according to the user's purpose. The analysis unit is implemented by, for example, the control unit 46A of the smart glasses 214, and analyzes an exercise form. The proposal unit is implemented by, for example, the specific processing unit 290 of the data processing apparatus 12, and proposes a meal plan. The management unit is implemented by, for example, the specific processing unit 290 of the data processing apparatus 12, and performs progress management. The image generation unit is implemented by, for example, the control unit 46A of the smart glasses 214, and presents “one's own appearance” before and after achieving the purpose using an image generation function. The correspondence between each unit and the device or the control unit is not limited to the above-described example, and various modifications are possible.

### Third Embodiment

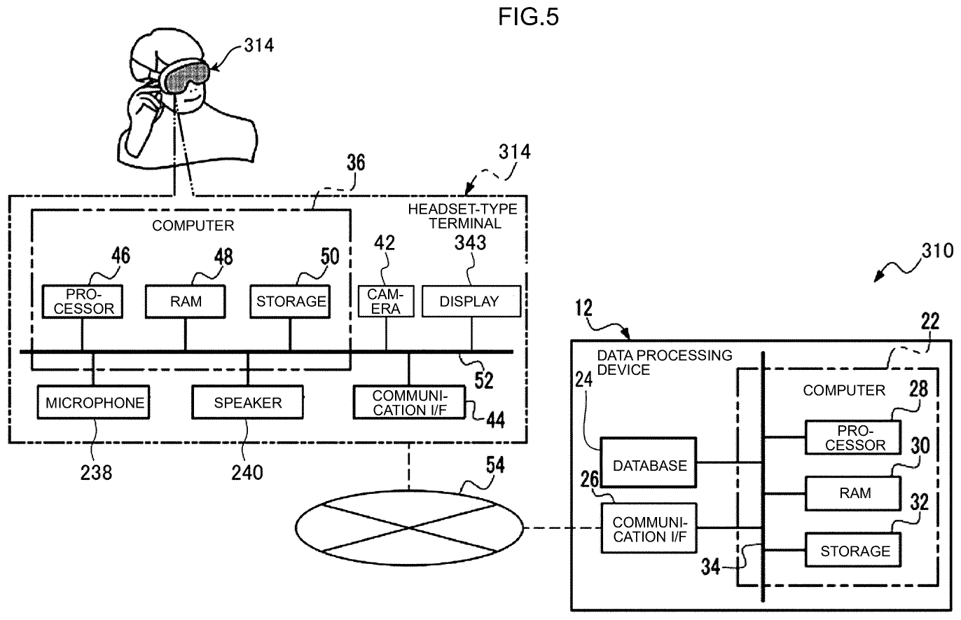

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

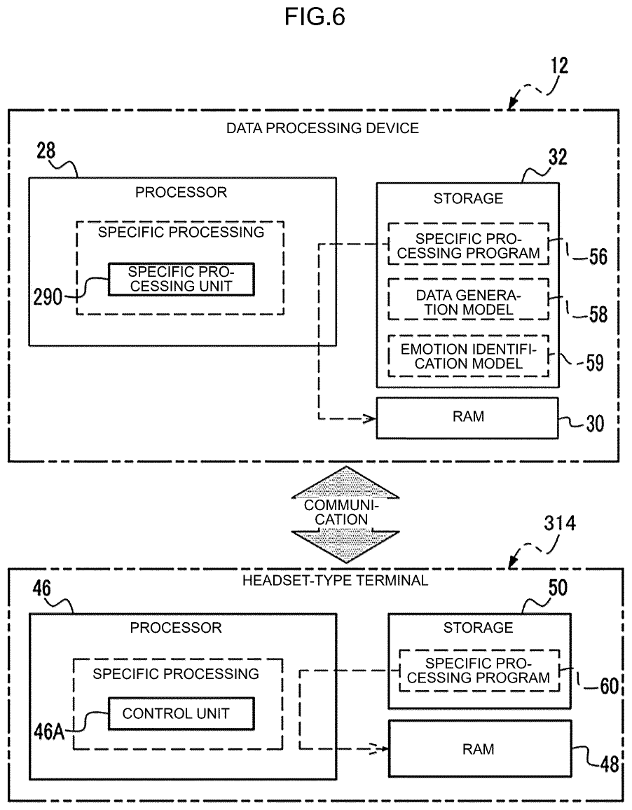

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described reception unit, generation unit, analysis unit, proposal unit, management unit, and image generation unit is implemented by, for example, at least one of the headset-type terminal 314 and the data processing apparatus 12. For example, the reception unit is implemented by a control unit 46A of the headset-type terminal 314, and a user inputs a training purpose. The generation unit is implemented by, for example, a specific processing unit 290 of the data processing apparatus 12, and generates a personal training menu according to the user's purpose. The analysis unit is implemented by, for example, the control unit 46A of the headset-type terminal 314, and analyzes an exercise form. The proposal unit is implemented by, for example, the specific processing unit 290 of the data processing apparatus 12, and proposes a meal plan. The management unit is implemented by, for example, the specific processing unit 290 of the data processing apparatus 12, and performs progress management. The image generation unit is implemented by, for example, the control unit 46A of the headset-type terminal 314, and presents “one's own appearance” before and after achieving the purpose using an image generation function. The correspondence between each unit and the device or the control unit is not limited to the above-described example, and various modifications are possible.

### Fourth Embodiment

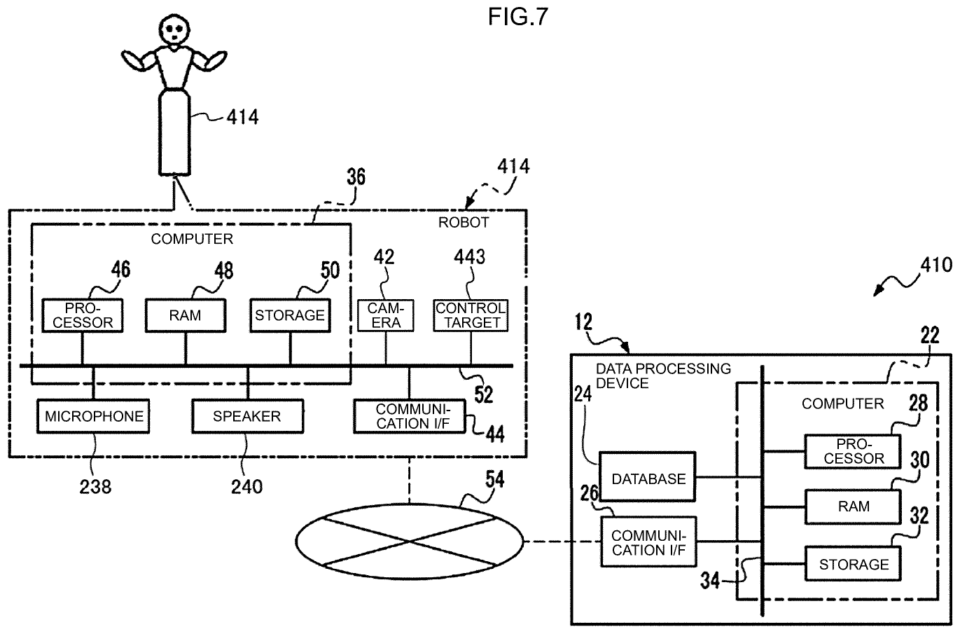

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

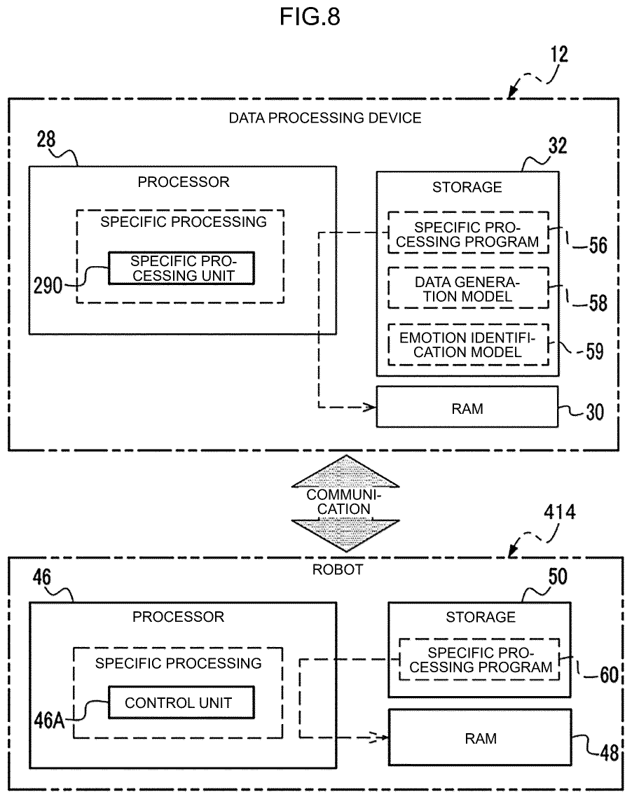

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described reception unit, generation unit, analysis unit, proposal unit, management unit, and image generation unit is implemented by, for example, at least one of the robot 414 and the data processing apparatus 12. For example, the reception unit is implemented by a control unit 46A of the robot 414, and a user inputs a training purpose. The generation unit is implemented by, for example, a specific processing unit 290 of the data processing apparatus 12, and generates a personal training menu according to the user's purpose. The analysis unit is implemented by, for example, the control unit 46A of the robot 414, and analyzes an exercise form. The proposal unit is implemented by, for example, the specific processing unit 290 of the data processing apparatus 12, and proposes a meal plan. The management unit is implemented by, for example, the specific processing unit 290 of the data processing apparatus 12, and performs progress management. The image generation unit is implemented by, for example, the control unit 46A of the robot 414, and presents “one's own appearance” before and after achieving the purpose using an image generation function. The correspondence between each unit and the device or the control unit is not limited to the above-described example, and various modifications are possible.

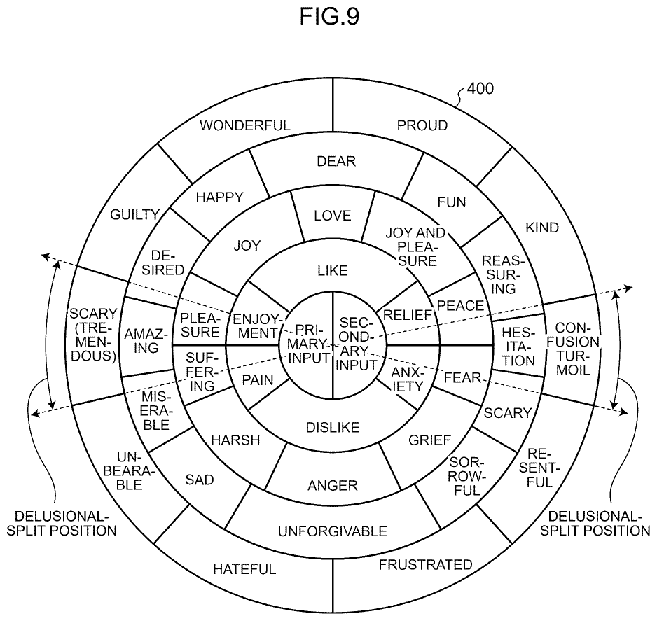

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

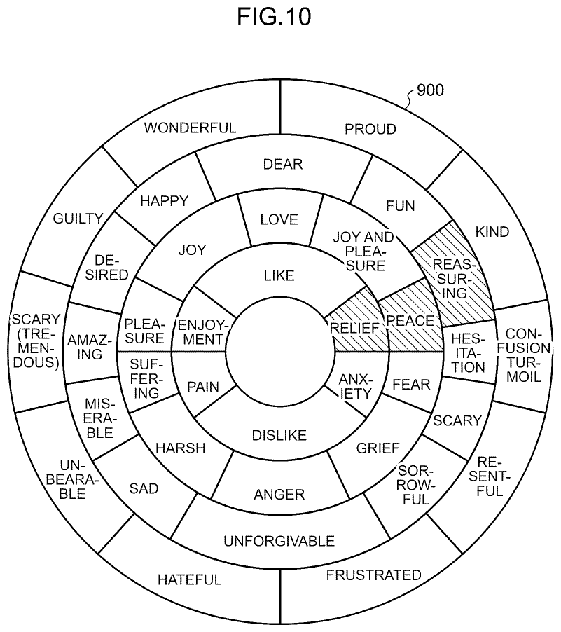

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a reception unit configured to receive a training purpose; a generation unit configured to generate a personal training menu based on the purpose received by the reception unit; an analysis unit configured to analyze an exercise form based on the menu generated by the generation unit; a proposal unit configured to propose a meal plan based on the menu generated by the generation unit; a management unit configured to manage progress based on the menu generated by the generation unit; and an image generation unit configured to present an appearance of a user before and after achieving the purpose using an image generation function.
(Supplementary Note 2) The system according to Supplementary Note 1, wherein the generation unit is configured to generate an exercise menu according to the user's purpose.
(Supplementary Note 3) The system according to Supplementary Note 1, wherein the analysis unit is configured to analyze a form of the user when the user performs an exercise, and confirm whether the exercise is being performed with an appropriate form.
(Supplementary Note 4) The system according to Supplementary Note 1, wherein the proposal unit is configured to generate a meal plan according to the user's purpose and provide the meal plan to the user.
(Supplementary Note 5) The system according to Supplementary Note 1, wherein the management unit is configured to manage progress of the user's training and provide support for achieving a goal.
(Supplementary Note 6) The system according to Supplementary Note 1, wherein the image generation unit is configured to generate images of an appearance of the user before and after starting the training, and present the images to the user.
(Supplementary Note 7) The system according to Supplementary Note 1, wherein the reception unit is configured to estimate an emotion of the user, and adjust an input method of the training purpose based on the estimated emotion of the user.
(Supplementary Note 8) The system according to Supplementary Note 1, wherein the reception unit is configured to analyze a past training history of the user, and propose a purpose input method.
(Supplementary Note 9) The system according to Supplementary Note 1, wherein the reception unit is configured to perform filtering based on a current health condition or lifestyle habits of the user when the training purpose is input.
(Supplementary Note 10) The system according to Supplementary Note 1, wherein the reception unit is configured to estimate an emotion of the user, and determine a priority of the input training purpose based on the estimated emotion of the user.
(Supplementary Note 11) The system according to Supplementary Note 1, wherein the reception unit is configured to consider geographical location information of the user when the training purpose is input, and prioritize input of a highly relevant purpose.
(Supplementary Note 12) The system according to Supplementary Note 1, wherein the reception unit is configured to analyze social media activity of the user when the training purpose is input, and propose a relevant purpose.
(Supplementary Note 13) The system according to Supplementary Note 1, wherein the generation unit is configured to estimate an emotion of the user, and adjust a generation method of the training menu based on the estimated emotion of the user.
(Supplementary Note 14) The system according to Supplementary Note 1, wherein the generation unit is configured to refer to past training data of the user when generating the training menu, and generate an optimal menu.
(Supplementary Note 15) The system according to Supplementary Note 1, wherein the generation unit is configured to customize the menu based on a health condition or lifestyle habits of the user when generating the training menu.
(Supplementary Note 16) The system according to Supplementary Note 1, wherein the generation unit is configured to estimate an emotion of the user, and determine a priority of the training menu based on the estimated emotion of the user.
(Supplementary Note 17) The system according to Supplementary Note 1, wherein the generation unit is configured to consider geographical location information of the user when generating the training menu, and generate an optimal menu.
(Supplementary Note 18) The system according to Supplementary Note 1, wherein the generation unit is configured to analyze social media activity of the user when generating the training menu, and customize the menu.
(Supplementary Note 19) The system according to Supplementary Note 1, wherein the analysis unit is configured to estimate an emotion of the user, and adjust an analysis method of the exercise form based on the estimated emotion of the user.
(Supplementary Note 20) The system according to Supplementary Note 1, wherein the analysis unit is configured to refer to past exercise data of the user when analyzing the exercise form, and improve accuracy of the analysis.
(Supplementary Note 21) The system according to Supplementary Note 1, wherein the analysis unit is configured to perform analysis based on a health condition or lifestyle habits of the user when analyzing the exercise form.
(Supplementary Note 22) The system according to Supplementary Note 1, wherein the analysis unit is configured to estimate an emotion of the user, and adjust a display method of an analysis result of the exercise form based on the estimated emotion of the user.
(Supplementary Note 23) The system according to Supplementary Note 1, wherein the analysis unit is configured to consider geographical location information of the user when analyzing the exercise form, and perform analysis.
(Supplementary Note 24) The system according to Supplementary Note 1, wherein the analysis unit is configured to analyze social media activity of the user when analyzing the exercise form, and propose points for improvement of the form.
(Supplementary Note 25) The system according to Supplementary Note 1, wherein the proposal unit is configured to estimate an emotion of the user, and adjust a proposal method of the meal plan based on the estimated emotion of the user.
(Supplementary Note 26) The system according to Supplementary Note 1, wherein the proposal unit is configured to refer to past meal data of the user when proposing the meal plan, and propose an optimal plan.
(Supplementary Note 27) The system according to Supplementary Note 1, wherein the proposal unit is configured to customize the plan based on a health condition or lifestyle habits of the user when proposing the meal plan.
(Supplementary Note 28) The system according to Supplementary Note 1, wherein the proposal unit is configured to estimate an emotion of the user, and determine a priority of the meal plan based on the estimated emotion of the user.
(Supplementary Note 29) The system according to Supplementary Note 1, wherein the proposal unit is configured to consider geographical location information of the user when proposing the meal plan, and propose an optimal plan.
(Supplementary Note 30) The system according to Supplementary Note 1, wherein the proposal unit is configured to analyze social media activity of the user when proposing the meal plan, and customize the plan.
(Supplementary Note 31) The system according to Supplementary Note 1, wherein the management unit is configured to estimate an emotion of the user, and adjust a method of progress management based on the estimated emotion of the user.
(Supplementary Note 32) The system according to Supplementary Note 1, wherein the management unit is configured to refer to past training data of the user during progress management, and improve accuracy of the management.
(Supplementary Note 33) The system according to Supplementary Note 1, wherein the management unit is configured to perform management based on a health condition or lifestyle habits of the user during progress management.
(Supplementary Note 34) The system according to Supplementary Note 1, wherein the management unit is configured to estimate an emotion of the user, and determine a priority of progress management based on the estimated emotion of the user.
(Supplementary Note 35) The system according to Supplementary Note 1, wherein the management unit is configured to consider geographical location information of the user during progress management, and perform management.
(Supplementary Note 36) The system according to Supplementary Note 1, wherein the management unit is configured to analyze social media activity of the user during progress management, and customize a method of the management.
(Supplementary Note 37) The system according to Supplementary Note 1, wherein the image generation unit is configured to estimate an emotion of the user, and adjust a method of image generation based on the estimated emotion of the user.
(Supplementary Note 38) The system according to Supplementary Note 1, wherein the image generation unit is configured to refer to past training data of the user during image generation, and improve accuracy of the generation.
(Supplementary Note 39) The system according to Supplementary Note 1, wherein the image generation unit is configured to customize the image based on a health condition or lifestyle habits of the user during image generation.
(Supplementary Note 40) The system according to Supplementary Note 1, wherein the image generation unit is configured to estimate an emotion of the user, and adjust a display method of the generated image based on the estimated emotion of the user.
(Supplementary Note 41) The system according to Supplementary Note 1, wherein the image generation unit is configured to consider geographical location information of the user during image generation, and generate the image.
(Supplementary Note 42) The system according to Supplementary Note 1, wherein the image generation unit is configured to analyze social media activity of the user during image generation, and customize the image.

What is claimed is:

1. A system comprising:
circuitry configured to:
receive, via a communication interface coupled to a packet-switched network, input data from a client terminal, the input data comprising at least one of text data or speech data;
generate a multidimensional tensor by encoding the input data using a Transformer-based neural network, and inputting the multidimensional tensor and a user attribute vector into a data generation model to output an inference data set comprising a plurality of scored entries;
extract, from continuous image frame data received from the client terminal, coordinate vectors of a plurality of skeletal key points by applying a convolutional neural network to each image frame, and computing a conformance score by comparing a time-series sequence of the coordinate vectors against a reference trajectory using a dynamic time warping algorithm;
generate a constrained output data set by executing a numerical optimization over structured records stored in a database, the numerical optimization minimizing an objective function subject to a plurality of constraints derived from the user attribute vector;
apply a long short-term memory network to historical time-series data associated with a user to generate a prediction value representing a future state;
generate a synthetic image by inputting a source image and a target attribute vector into a generative adversarial network, the generative adversarial network performing a vector operation on a latent representation extracted from the source image by an encoder, conditioned on the target attribute vector, and reconstructing the synthetic image through a decoder; and
transmit the inference data set, the conformance score, the constrained output data set, the prediction value, and the synthetic image to the client terminal via the communication interface and the packet-switched network.

2. The system according to claim 1, wherein the input data comprises a training purpose associated with at least one of health improvement, weight reduction, or muscle strength improvement, and wherein the inference data set comprises a personalized activity menu comprising a plurality of activity items each associated with a respective scored entry.

3. The system according to claim 2, wherein the data generation model comprises a hybrid recommendation algorithm combining collaborative filtering and content-based filtering, and wherein the user attribute vector comprises at least one of an age value, a weight value, a body composition value, or an exercise history vector.

4. The system according to claim 1, wherein the convolutional neural network comprises a backbone architecture selected from a group consisting of ResNet and EfficientNet, and wherein the coordinate vectors comprise two-dimensional or three-dimensional coordinates of 17 to 33 joint points of a human body together with confidence scores for each joint point.

5. The system according to claim 1, wherein the circuitry is further configured to input the time-series sequence of the coordinate vectors into a temporal convolutional network or a long short-term memory network to compute a cycle, a velocity, an acceleration, and a change pattern of joint angles of a motion represented by the coordinate vectors.

6. The system according to claim 1, wherein the dynamic time warping algorithm compares the time-series sequence of the coordinate vectors against an ideal trajectory model learned from expert motion data, and wherein the conformance score comprises at least one of a cosine similarity score or a Euclidean distance score.

7. The system according to claim 1, wherein the constrained output data set comprises a nutrient-optimized plan, and wherein the numerical optimization is formulated as a constraint satisfaction problem with an objective function defined as maximization of a user preference score and minimization of an error from target nutrient values, the target nutrient values comprising a target calorie intake and a target ratio of macronutrients.

8. The system according to claim 1, wherein the circuitry is further configured to receive, from the client terminal, feedback data comprising at least one of image data or text data representing an action performed by the user, and to perform dynamic recalculation of the constrained output data set to compensate for a deviation identified from the feedback data.

9. The system according to claim 1, wherein the long short-term memory network receives, as the historical time-series data, multivariate time-series data comprising at least one of daily activity result values, physical measurement values, or subjective fatigue level values, and wherein the prediction value comprises a predicted goal achievement date.

10. The system according to claim 1, wherein the circuitry is further configured to detect a plateau in the historical time-series data by analyzing a trend using at least one of a moving average, exponential smoothing, or an ARIMA model, and to transmit a feedback control signal to adjust a parameter of the data generation model in response to detecting the plateau.

11. The system according to claim 1, wherein the generative adversarial network comprises an encoder that converts the source image into a latent vector in a latent space, and wherein the vector operation comprises identifying a direction vector corresponding to the target attribute vector in the latent space and moving the latent vector in the identified direction, and wherein the decoder reconstructs a high-resolution RGB image from the manipulated latent vector while maintaining identity features of the source image.

12. The system according to claim 1, wherein the circuitry is further configured to generate a plurality of intermediate synthetic images representing progressive states between a current state of the source image and a target state defined by the target attribute vector, and to transmit the plurality of intermediate synthetic images to the client terminal as a morphing sequence.

13. The system according to claim 1, wherein the circuitry is further configured to estimate an emotion of the user by applying an emotion identification model to multimodal sensor data received from the client terminal, the multimodal sensor data comprising at least one of voice waveform data, facial image data, or text input pattern data, and to adjust a parameter of at least one of the data generation model, the convolutional neural network, or the generative adversarial network based on the estimated emotion.

14. The system according to claim 13, wherein the emotion identification model comprises a multimodal Transformer that receives prosodic features extracted from the voice waveform data, facial muscle movement features extracted from the facial image data, and emotion polarity features extracted from the text input pattern data, and outputs a probability distribution over a plurality of emotion categories.

15. The system according to claim 13, wherein the circuitry is further configured to adjust a display format of the inference data set transmitted to the client terminal based on the estimated emotion, such that when the estimated emotion indicates stress, the circuitry generates the inference data set in a simplified format, and when the estimated emotion indicates relaxation, the circuitry generates the inference data set in a detailed format.

16. The system according to claim 1, wherein the circuitry is further configured to receive geographic location information from the client terminal, identify a facility category associated with the geographic location information by querying a point-of-interest database using a spatial index, and filter the plurality of scored entries in the inference data set based on the identified facility category.

17. The system according to claim 1, wherein the circuitry is further configured to analyze past input data and past inference data sets stored in the database to compute a conditional probability of a next input selection by the user using at least one of a Markov chain model or a recurrent neural network, and to transmit a predicted input selection to the client terminal.

18. A system comprising:
circuitry configured to:
receive, via a communication interface coupled to a packet-switched network, input data from a client terminal, the input data comprising at least one of text data or speech data;
convert the input data into a high-dimensional embedding vector by applying a Transformer-based language model, and extract an intent classification and a set of parameters from the embedding vector;
generate a multidimensional tensor by combining the extracted intent classification, the set of parameters, and a user attribute vector comprising at least one of an age value, a weight value, or a body composition value, and input the multidimensional tensor into a data generation model comprising a neural network to output an inference data set, the inference data set comprising a plurality of entries each associated with a fitness score ranging from 0 to 1;
receive continuous image frame data from the client terminal via the communication interface, the continuous image frame data comprising an RGB image tensor, apply a convolutional neural network having a ResNet or EfficientNet backbone to each image frame to extract two-dimensional or three-dimensional coordinate vectors of a plurality of skeletal key points, input a time-series sequence of the coordinate vectors into a temporal convolutional network to compute joint angle change patterns, and calculate a conformance score by applying a dynamic time warping algorithm to compare the time-series sequence against an ideal trajectory model;
calculate target nutrient values comprising a target calorie intake and a target macronutrient ratio from the user attribute vector, and generate a constrained output data set by executing a constraint satisfaction optimization over structured records stored in a database, the constraint satisfaction optimization maximizing a user preference score while minimizing an error from the target nutrient values;
apply a long short-term memory network to multivariate historical time-series data associated with a user, the multivariate historical time-series data comprising daily activity result values and physical measurement values, to generate a prediction value comprising a predicted goal achievement date, and detect a plateau in the multivariate historical time-series data by analyzing a trend;
receive a source image of the user from the client terminal, extract a latent vector from the source image using an encoder of a generative adversarial network, perform a vector operation on the latent vector in a latent space by moving the latent vector in a direction corresponding to a target attribute vector, and reconstruct a synthetic image through a decoder of the generative adversarial network, the synthetic image maintaining identity features of the source image while reflecting the target attribute vector; and
transmit the inference data set, the conformance score, the constrained output data set, the prediction value, and the synthetic image to the client terminal via the communication interface and the packet-switched network.

19. The system according to claim 18, wherein the circuitry is further configured to estimate an emotion of the user by applying an emotion identification model comprising a multimodal Transformer to at least one of voice waveform data, facial image data, or text input pattern data received from the client terminal, and to adjust at least one of a generation parameter of the data generation model, a rendering parameter of the generative adversarial network, or a display format of the inference data set based on the estimated emotion.

20. A method performed by circuitry of a system, the method comprising:
receiving, via a communication interface coupled to a packet-switched network, input data from a client terminal, the input data comprising at least one of text data or speech data;
generating a multidimensional tensor by encoding the input data using a Transformer-based neural network, and inputting the multidimensional tensor and a user attribute vector into a data generation model to output an inference data set comprising a plurality of scored entries;
extracting, from continuous image frame data received from the client terminal, coordinate vectors of a plurality of skeletal key points by applying a convolutional neural network to each image frame, and computing a conformance score by comparing a time-series sequence of the coordinate vectors against a reference trajectory using a dynamic time warping algorithm;
generating a constrained output data set by executing a numerical optimization over structured records stored in a database, the numerical optimization minimizing an objective function subject to a plurality of constraints derived from the user attribute vector;
applying a long short-term memory network to historical time-series data associated with a user to generate a prediction value representing a future state;
generating a synthetic image by inputting a source image and a target attribute vector into a generative adversarial network, the generative adversarial network performing a vector operation on a latent representation extracted from the source image by an encoder, conditioned on the target attribute vector, and reconstructing the synthetic image through a decoder; and
transmitting the inference data set, the conformance score, the constrained output data set, the prediction value, and the synthetic image to the client terminal via the communication interface and the packet-switched network.
